(19) United States
(12) Patent Application Publication
(10) Pub. No.: US 2013/0323165 A1
(43) Pub. Date: Dec. 5, 2013

(54) Magnetic Nanoplatforms for Theranostic and Multi-Modal Imaging Applications

(71) Applicants: Robert B. Campbell, Quincy, MA (US); Srinivas Sridhar, Newton, MA (US)

(72) Inventors: Robert B. Campbell, Quincy, MA (US); Srinivas Sridhar, Newton, MA (US)

(73) Assignee: NORTHEASTERN UNIVERSITY, Boston, MA (US)

(21) Appl. No.: 13/583,616

(22) PCT Filed: Mar. 8, 2011
(86) PCT No.: PCT/US11/27573

§ 371 Date: Aug. 22, 2013

Related U.S. Application Data
(60) Provisional application No. 61/311,697, filed on Mar. 8, 2010.

(51) Int. Cl. A61K 9/127; A61N 2/02

(52) U.S. Cl. CPC A61K 9/1272; A61N 2/02
USPC 424/1.21; 424/9.3; 424/1.11; 424/9.6; 424/9.1; 424/450; 514/44 A; 600/12

(57) ABSTRACT

Disclosed are nanoparticle compositions comprising paramagnetic particles, radiolabels, fluorophores, and/or positron emission tomography agents encapsulated within a biocompatible vehicle. In addition, methods of multi-modal diagnostic imaging and treating diseased tissues are disclosed, wherein the methods comprises administering a nanoparticle composition to a subject in which the nanoparticle composition comprises paramagnetic particles, radiolabels, fluorophores, and positron emission tomography agents encapsulated within a biocompatible vehicle.

This Application claims the benefit of priority to U.S. Provisional Application No. 61/311,697, filed Mar. 8, 2010, the specification of which is incorporated by reference in its entirety.

### FIELD OF THE INVENTION

The present invention disclosure is in the field of medicinal delivery of nanoparticles. More particularly, the present disclosure relates to the preparation and use of magnetic cationic liposomal nanoparticles.

### BACKGROUND

More than 23% of all human deaths in the United States are associated with cancer (Pitot. “Cancer—An overview,” Cancer: The Outlaw Cell. E. R. Lafond (ed.), 2nd Ed. (pp. 1-18). American Chemical Society, Washington, 1988). The development of effective treatment strategies is therefore of vital importance, and is urgently needed.

One clinical approach targets functional tumor vessels in an effort to destroy the existing tumor vasculature. A common goal of all successful vascular targeting strategies is the ability to interrupt the flow of oxygen and nutrients to the developing tumor mass. Tumor vessels are the main focus of this therapeutic approach, and cancer cells die as a result of vascular injury (Chaplin, et al. (1999) Br. J. Cancer, 80(1): 57-64).

One potential problem associated with the use of vascular targeting strategies is the lack of specificity. For this reason, the ability to reduce the total amount of drug delivered to healthy tissues, while improving selective delivery to tumor targets is a formidable challenge. Success with chemotherapy is thus dependent on the correct identification of therapeutic agents, as well as accurate identification and appropriate use of strategies to deliver them to targets (Alexiou, et al. (2000) Cancer Res., 60(23):6641-8).

Another contribution to the field of targeted drug delivery involves the use of cationic liposomes (Campbell, et al. (2002) Cancer Res., 62:6831-6836; Kunstfeld, et al. (2003) J. Invest. Dermatol. 120:476-482; Strieth, et al., (2004) Int. J. Cancer, 110:117-124; Thurston, et al. (1998) J. Clin. Invest., 101:1401-1413). Cationic liposomes have been shown to target tumor vessels to a significant extent over vessels in normal healthy tissues, targeting approximately 25 and 5% of vessel areas respectively Although cationic liposomes accumulate in tumors, the distribution of liposomes along tumor vessels is non-uniform, and although many vessels are targeted some vessel areas are not targeted by this approach. There is a need for a theranostic platform that will simultaneously combine the modalities of targeting, imaging agent and therapeutic delivery to the disease site.

### SUMMARY

This disclosure relates to the improved distribution of biocompatible vehicles, such as cationic liposomes, along the tumor vasculature with use of an externally applied magnetic field. The disclosure also relates to multimodal imaging of tissues using biocompatible vehicles localized to those tissues and allowing for imaging by multiple types of techniques.

This disclosure comprises a nanoparticle composition, such as a liposome, that acts as a theranostic platform. These nanoparticle compositions are multi-functional, can be biodegradable and clear out of the body with minimum toxicity, have targeting capability, and carry a variety of cargos including diagnostic, imaging and therapeutic agents; target the tumor with very high specificity. Their use enables imaging through magnetic contrast enhancement and delivery of therapeutic agent at the tumor site “on-demand” or with tailored release profile.

Aspects disclosed herein relate to nanoparticle compositions comprising a biocompatible vehicle encapsulating three or more of the group consisting of paramagnetic particles, radiolabels, fluorophores, and positron emission tomography agents, the nanoparticle composition being from about 30 nm to about 250 nm. As used herein, the term “encapsulate” means to enclose molecules within a structure. The term is meant to encompass instances where a molecule is located within a membrane such as a lipid membrane. It is also meant to encompass embodiments where the molecule is located within an aqueous environment of a vesicle, e.g., within a micelle or liposome.

Further aspects disclosed herein relate to methods of multi-modal diagnostic imaging. The methods comprise administering a nanoparticle composition to a subject, the nanoparticle composition comprising a biocompatible vehicle encapsulating three or more of the group consisting of paramagnetic particles, radiolabels, fluorophores, and positron emission tomography agents. The methods further comprise allowing the nanoparticle composition to bind to a tissue or to circulate in the vasculature in the subject and detecting the nanoparticle composition by one or more imaging techniques selected from the group consisting of positron emission tomography (PET), magnetic resonance imaging (MRI), single photon emission computed tomography (SPECT/CT), and optical imaging. The methods also comprise generating one or more images of the tissue bound by the nanoparticle composition or of the circulation system and registering the images from different modalities to obtain accurate location of the tissue and or the organs being imaged.

In additional aspects, methods of treating diseased tissue are disclosed. In certain aspects, the methods comprise administering a nanoparticle composition comprising a biocompatible vehicle encapsulating two or more of the group consisting of paramagnetic particles, radiolabels, fluorophores, and positron emission tomography agents and allowing the nanoparticle composition to bind to a diseased tissue in the subject, wherein the nanoparticle composition localizes to the diseased tissue. The methods also comprise subjecting the nanoparticle composition to a magnetic field such that the temperature of the nanoparticle composition increases; thereby killing the diseased tissue. In certain embodiments, the methods entail subjecting the nanoparticle composition to an alternating (ac) magnetic field such that the temperature of the nanoparticle composition and attached tissue increases to 42-45° C. for hyperthermic treatment, and above 45° C. for thermal ablation; thereby killing the diseased cells in the tissue.

In other aspects, the methods comprise administering a nanoparticle composition comprising a biocompatible vehicle encapsulating paramagnetic particle and siRNA molecules. Such aspects also comprise allowing the nanoparticle composition to localize to a diseased tissue in the subject, the siRNA molecules being released into the cells of the diseased tissue so that the diseased tissue is treated with the siRNA.

### BRIEF DESCRIPTION OF THE FIGURES

FIG. 1A is a graphic representation showing the effect of different concentrations (mg/ml) of MAG-C on size of liposomes, with increasing MAG-C leading to increased size) (nm).

FIG. 1B is a graphic representation showing the effect of different concentrations (mg/ml) of MAG-C on zeta potential of liposomes.

FIG. 1C is a graphic representation showing the effect of different concentrations (mg/ml) of MAG-C on phase transition temperature (° C.) of liposomes.

FIG. 1D is a pictorial representation of a liposome showing lipid-soluble drugs encapsulated in the liposome membrane, water-soluble drugs encapsulated in the liposomal aqueous interior, and MAG-C distributed throughout.

FIG. 2 is a graphic representation showing the association of MAG-C cationic liposomes with melanoma and endothelial cells in vitro. Cells (B16-F10, squares; HTB-72, open circles; HMVEC-d, triangles) were seeded at 1×10⁴ cells/ml in a 48 well plate and incubated at 37° C. The relative association of cells with each liposome preparation type was determined 24 h following cell exposure to rhodamine labeled liposomes (10-1000 nmol). The control group was untreated. Each value represents the mean±S.D. of 6 different determinations.

FIG. 3A is a pictorial representation showing Analysis of MAG-C association with liposomes. Liposomes were prepared as discussed in the Examples. Images were acquired by DIC microscopy and RGB. Incorporation of MAG-C in cationic liposomes: (i) cationic liposomes (DMPC/DMTAP/cholesterol) under DIC (40×), (ii) DIC image of MAG-C cationic liposomes under RGB filter (100×), (iii) DIC image of MAG-C cationic liposomes taken up by B16-F10 cells under RGB filter (40×). Arrows indicate MAG-C.

FIG. 3B is a pictorial representation showing the intracellular uptake of MAG-C cationic liposomes: Cells were seeded at 5×105 cells/ml in a 6 well plate. Cells were treated with rhodamine labeled MAG-C cationic liposomes for 24 h at 37° C. with 100 nmol of liposomes. Rhodamine labeled MAG-C cationic liposomes are indicated in red. The blended image of fluorescence and DIC show the localization of MAG-C in cells. Magnification setting for B16-F10 and HTB-72 was 20×, and for HMVEC-d was 40×.

FIG. 4A is a graphical representation showing cell association using B16-F10 cells—comparison of MAG-C cationic liposomes versus PEGylated cationic liposomes (PCLs) containing MAG-C cells were seeded at 1×10⁴ cells/ml in a 48 well plate and incubated at 37° C. Association measurements were determined 24 h following exposure of cells to various amounts of rhodamine labeled MAG-C cationic liposomes, and MAG-C PEGylated cationic liposomes (10-1000 nmol). The control group was untreated. MAG-C PEGylated cationic liposomes interacted with all three cell lines to a significantly less extent compared to MAG-C cationic liposomes without the inclusion of PEG. Each value represents the mean±S.D. of 3 sets of determination, P<0.01.

FIG. 4B is a graphical representation showing cell association using HTB-72 cells—comparison of MAG-C cationic liposomes versus PEGylated cationic liposomes (PCLs) containing MAG-C cells were seeded at 1×10⁴ cells/ml in a 48 well plate and incubated at 37° C. Association measurements were determined 24 h following exposure of cells to various amounts of rhodamine labeled MAG-C cationic liposomes, and MAG-C PEGylated cationic liposomes (10-1000 nmol). The control group was untreated. MAG-C PEGylated cationic liposomes interacted with all three cell lines to a significantly less extent compared to MAG-C cationic liposomes without the inclusion of PEG. Each value represents the mean±S.D. of 3 sets of determination, P<0.01.

FIG. 4B is a graphical representation showing cell association using HUVEC cells—comparison of MAG-C cationic liposomes versus PEGylated cationic liposomes (PCLs) containing MAG-C cells were seeded at 1×10⁴ cells/ml in a 48 well plate and incubated at 37° C. Association measurements were determined 24 h following exposure of cells to various amounts of rhodamine labeled MAG-C cationic liposomes, and MAG-C PEGylated cationic liposomes (10-1000 nmol). The control group was untreated. MAG-C PEGylated cationic liposomes interacted with all three cell lines to a significantly less extent compared to MAG-C cationic liposomes without the inclusion of PEG. Each value represents the mean±S.D. of 3 sets of determination, P<0.01.

FIG. 5A is a graphical representation showing the mean % D/G for DMPC/DMTA P/CHOL/MAG-C (clear columns) and DMPC/DMTA P/CHOL/PEG/MAG-C (dark columns) in liver, lung, and spleen.

FIG. 5B is a graphical representation showing the mean tumor/blood ratio for DMPC/DMTA P/CHOL/MAG-C (clear columns) and DMPC/DMTA P/CHOL/PEG/MAG-C (dark columns).

FIG. 6 is a graphical representation showing iron content in different liposomal formulations. MAG-C liposomes containing either 0.5 or 2.5 mg/ml were prepared as described in the Examples.

FIG. 7A is a graphical representation showing the percent of (3 mol %) etoposide loaded in MAG-C liposomes (MAG-C liposomes, bars and PEGylated MAG-C liposomes, checkered) containing 0, 0.5, 2.5 mg/ml of MAG-C.

FIG. 7B is a graphical representation showing the percent of (3 mol %) vinblastine sulfate loaded in MAG-C liposomes (MAG-C liposomes, bars and PEGylated MAG-C liposomes, checkered) containing 0, 0.5, 2.5 mg/mi of MAG-C.

FIG. 7C is a graphical representation showing the percent of (3 mol %) dacarbazine loaded in MAG-C liposomes (MAG-C liposomes, bars and PEGylated MAG-C liposomes, checkered) containing 0, 0.5, 2.5 mg/ml of MAG-C.

FIG. 8A is a graphical representation showing squid magnetization versus magnetic field values for cationic liposomes alone.

FIG. 8B is a graphical representation showing squid magnetization versus magnetic field values for cationic liposomes containing 0.5 mg/ml MAG-C.

FIG. 8C is a graphical representation showing squid magnetization versus magnetic field values for cationic liposomes containing 2.5 mg/ml MAG-C.

FIG. 8D is a graphical representation showing saturation magnetization MAG-C cationic liposomes containing 0.5 mg/ml MAG-C compared with MAG-C cationic liposomes containing 2.5 mg/ml MAG-C.

FIG. 9 is a graphical representation showing the effect of external magnetic field on tumor distribution of MAG-C cationic liposomes. Approximately 2×10⁶ B16-F10 cells were injected

subcutaneously in mice bearing melanoma tumors of approximately 250 nm3 volume. Mice were injected with ¹¹¹In labeled MAG-C cationic liposomes and external magnet of strength 1.2 T was placed for 1 h on the external surface of tumor mass. After 1 and 2 h post injection, radioactivity in tumor was measured; data are expressed as percent of label recovered per/gram of tumor (see Experimetal procedures). Each value represents mean±S.D. of 4 animals, P<0.05.

FIG. 10A is a graphical representation showing SQUID magnetization emu/g) vs. Field (Oe) of cationic liposomes without SPION showing diamagnetic response.

FIG. 10B is a graphical representation showing SQUID magnetization (emu/g) vs. Field (Oe) of MCL showing superparamagnetic response.

FIG. 11A is a pictorial representation showing TEM images of cationic liposomes alone.

FIG. 11B is a pictorial representation of TEM Images of MAG-C liposomes containing 0.5 mg/ml of SPION.

FIG. 11C is a pictorial representation showing TEM Images of MAG-C liposomes containing 2.5 mg/ml of SPION.

### DESCRIPTION

### 1. General

All publications, patent applications, patents, and other references mentioned herein are incorporated by reference in their entirety. In case of conflict, the present specification, including definitions, will control. In addition, the materials, methods, and examples are illustrative only and not intended to be limiting. Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below.

Nanotechnology provides a unique and unprecedented opportunity to develop multi-functional platforms that can achieve targeting to the disease site, deliver therapeutic agents, while also providing enhanced imaging capabilities for diagnosis and monitoring of progress and impact of the therapy. These theranostic platforms have the potential to open up a new era of personalized medicine in oncology. The challenge is to achieve these benefits while at the same time avoiding toxicity. Magnetic Cationic Liposomes (MCLs) are an excellent platform that can carry payloads to achieve all the ideal characteristics of a theranostic agent. Their cationic nature preferentially targets the tumor vasculature and binds to the tumor walls; the magnetic cargo is highly efficacious in enabling magnetic guidance to achieve enhanced tumor accumulation, as well as enabling imaging as a MRI contrast enhancement agent, and the cargo can include anticancer drugs which can be released either in a sustained manner, or “on-demand” through an external triggering agent.

The construct is comprised of a lipid bilayer which can enclose water and water-soluble chemicals and hydrophobic entities incorporated into the lipid bilayer. The construct can act as a container for targeting agents including but not limited to antibodies, ligands, targeting molecules, and also magnetic nanoparticles for magnetic guidance targeting; image contrast enhancement agents such as magnetic nanoparticles, fluorescent molecules, quantum dots and metallic nanoparticles for imaging techniques including but not limited to MRI, optical imaging, X-ray and other imaging techniques; and therapeutic agents including but not limited to anti-cancer drugs, anti-infectives, and other chemical entities, as well as entities that will couple to external agencies like alternating magnetic fields and electromagnetic radiation (microwaves, light, etc).

A prerequisite for any successful magnetic drug targeting approach is that the ferro fluid-drug complex must reach the tumor microcirculation and release the drug at this location. One exemplary approach is magnetic drug targeting (MDT) (Alexiou, et al. (2000) Cancer Res., 60:6641-6648; Alexiou, et al., (2003) J. Drug Target, 11:139-149; Zhang, et al. (2005) Pharm. Res., 22:573-583). MDT selectively delivers chemotherapeutic agents to tumors with the use of ferro fluids bound to drugs that can respond to an external magnetic field (Alexiou, et al. (2003) J. Drug Target, 11:139-149; Babincova, et al. (2004) Med. Phys., 31:2219-2221). In certain embodiments, magnetic targeting is accomplished using direct current magnetic fields. The fields can be applied for 1 or more hours depending on the size of the tumor, the type of tumor, and the amount of MNL administered.

In the MDT strategy, the magnetic field retains the chemotherapeutic agent at the intended site of drug action, and therefore increases drug levels at this location. This minimizes the potential for accumulation of drug in healthy tissues. Furthermore, the combination of improved target selectivity, and enhanced duration of drug exposure to target consequently reduce the overall amount of drug taken up by the RES (reticuloendothelial system). (Alexiou, et al. (2000) Cancer Res., 60:6641-6648).

Parameters to be considered for magnetic drug targeting are: (1) the concentration, and type of ferro fluid employed, (2) the magnetic strength of the external magnetic field, (3) and the length of time the target tissue is exposed to the external magnet. All three parameters should be carefully selected and optimized for each purpose (Alexiou, et al. (2003) J. Drug Target, 11:139-149; Babincova, et al. (2000) Z. Naturforsch [C], 55:278-281).

The biocompatible vehicles (i.e., delivery vehicles) disclosed herein have the characteristics of: (I) they are relatively tumor specific; (2) are capable of interacting with tumor vessels; (3) and are capable of responding to an external magnetic field. Biocompatible vehicles such as the cationic liposomes disclosed herein improve tumor vascular interactions and interact uniformly with the tumor vasculature.

In particular aspects, the biocompatible vehicles comprise magnetic structures. The magnetic structures allow for the localization, imaging, and treatment of diseased tissues and/or diseased cells within tissues. Magnetic structures can alter the MRI signal in their vicinity and hence can lead to differentiation of local environments that contain them from those that do not. Some contrast enhancement agents employ iron oxide (IO) or Gd based complexes. A variety of other magnetic entities have been developed. Superparamagnetic iron oxide nanoparticles (“SPIONs”) have been packaged in a variety of platforms including polymeric nanoparticles, micelles, dendrimers, fullerenes and liposomes. SPIONs are useful in the presently disclosed methods and nanoparticle compositions. SPIONs allow for the localization of nanoparticle compositions and the treatment of diseased tissues.

### 2. Nanoparticle Compositions

Disclosed herein are nanoparticle compositions comprising a biocompatible vehicle. The biocompatible vehicle (e.g., delivery vehicle) allows for the delivery of agents to target tissues. Target tissues include organ tissues from organs such as liver, heart, brain, skin, lung, stomach, and intestines. Tissues also include blood and bone.

In particular embodiments, the target tissues are diseased. The tissues that are diseased can be composed of healthy cells and diseased cells. Diseased cells can be cells that are precancerous or cancerous, i.e., cells that exhibit the features of a cancer cell. In particular embodiments, a cancer cell in a diseased tissue can be a breast cancer cell, an ovarian cancer cell, a myeloma cancer cell, a lymphoma cancer cell, a melanoma cancer cell, a sarcoma cancer cell, a leukemia cancer cell, a retinoblastoma cancer cell, a hepatoma cancer cell, a glioma cancer cell, a mesothelioma cancer cell, or a carcinoma cancer cell. In addition, diseased tissues can be infected with a virus or bacteria.

In certain aspects, the disclosed nanoparticle compositions are liposomes. Liposomes are artificial membrane-bound vesicles. Liposomal membranes are composed of phospholipids such as N-1-[1-(2,3-Dioleoyloxy)propyl]-N,N,Ntrimethylammonium methylsulfate (DOTAP), dimyristoyltrimethylammonium propane (DMTAP) and dipalmitoylphosphatidylcholine (DPPC) and/or dimyristoylphosphatidylcholine (DMPC). Liposomes come in a variety of types: (1) multilamellar vesicles; (2) small unilamellar vesicles; and (3) large unilamellar vesicles. Liposomes are well known for their ability to deliver drugs and other agents to a target.

Methods of forming liposomal formulations are known in the art. For example, liposomes can be formed by combining agents (e.g., therapeutic and imaging) disclosed herein and a phosphatidyl glycerol lipid derivative (PGL derivative). Briefly, agents and PGL derivative are mixed in a range of 1:1 to 1:2.1 to form a liposome/agent mixture. Alternatively, the ratio of agents to PGL derivative is in the ranges 1:1.2; or 1:1.4; or 1:1.5; or 1:1.6; or 1:1.8 or 1:1.9 or 1:2.0 or 1:2.1. The mixture is then combined with an effective amount of at least a 20% organic solvent such as an ethanol solution to form liposomes containing the agents.

Liposomal formulations can be prepared by a thin film and hydration method. In such methods, a rotary evaporator is employed to remove solvent from a pyrex tube containing lipid mixed at the appropriate ratios and the purex tube (placed inside a round bottom flask) rotated continuously in the water bath at 42° C. for 30 minutes or until a thin film is deposited on the inside wall of pyrex tube. The lipid film is hydrated with PBS and then is placed in an ice bucket at 15-minute intervals for at least 8 cycles. To reduce particle size, liposomes are passed through a 0.1 μm filter for 11 times by extrusion (Avanti Polar Lipids, Alabaster, Ala.). Particle size and zeta (ζ) potential of liposomes after extrusion are determined by 90 Plus Particle/Zeta Potential Analyzer (Brookhaven Instruments, Holtsville, N.Y.).

Embodiments of the disclosed nanoparticle compositions include cationic liposomes. Cationic liposomes have been shown to mediate intracellular delivery of nucleic acids, such as plasmid DNA (Felgner et al., Proc. Natl. Acad. Sci. USA (1987) 84:7413-7416, which is herein incorporated by reference); mRNA (Malone et al., Proc. Natl. Acad. Sci. USA (1989) 86:6077-6081, which is herein incorporated by reference); and purified transcription factors (Debs et al., J. Biol. Chem. (1990) 265:10189-10192, which is herein incorporated by reference), in functional form.

Cationic liposomes are readily available. For example, N[1-2,3-dioleyloxy)propyl]-N,N,N-triethylammonium (DOTMA) liposomes are particularly useful and are available under the trademark Lipofectin, from GIBCO BRL, Grand Island, N.Y. (See also Feigner et al., Proc. Natl. Acad. Sci. USA (1987) 84:7413-7416, which is herein incorporated by reference). Other commercially available liposomes include transfectace (DDAB/DOPE) and DOTAP/DOPE (Boehringer).

Other cationic liposomes can be prepared from readily available materials using techniques well known in the art. See, e.g. PCT Publication No. WO 90/11092 (which is herein incorporated by reference) for a description of the synthesis of DOTAP (1,2-bis(oleoyloxy)-3-(trimethylammonio)propane)liposomes. Preparation of DOTMA liposomes is explained in the literature, see, e.g., P. Felgner et al., Proc. Natl. Acad. Sci. USA 84:7413-7417, which is herein incorporated by reference. Similar methods can be used to prepare liposomes from other cationic lipid materials.

In order to achieve maximum therapeutic efficacy by avoiding rapid clearance from the blood circulation by the reticuloendothelial system (RES), liposomal formulations can incorporate components such as polyethylene glycol (PEG) (see Klibanov et al. FEBS Lett. 268: 235-7 (1990); Mayuryama et al. Biochim. Biophys. Acta 1128: 44-49 (1992); Allen et al. Biochim. Biophys. Acta 1066: 29-36 (1991)). PEG conjugation to liposomes such as immunoliposomes has been shown to prolong liposome circulation in blood, as well as to enhance the therapeutic efficacy of liposomal drugs (Daemen et al. J. Control Rel. 44: 1-9 (1997); Storm et al. Clin. Cancer Res. 4: 111-115 (1998); Vaage et al. Br. J. Cancer 75: 482-6 (1997); Gabizon et al. Cancer Res. 54: 987-92 (1994)). In certain instances, it is advantageous to place cell-specific antibodies at the distal end of the PEG polymer to obtain efficient target binding by avoiding steric hindrance from the PEG chains. This type of immunoliposome formulation has been used successfully for in vivo targeting to the lungs (Maruyama et al. Biochim. Biophys. Acta 1234: 74-80 (1995)). In particular embodiments, liposomes have PEG attached to their surface. In some embodiments, the PEG is further conjugated to a particle (e.g., gold), protein (e.g., lectin, ligands, receptors), or fatty acid (see, e.g., Bakowski et al. Biochimica et Biophysica Acta (BRA)—Biomembranes, 1778(1):242-249 (2008); Melzak et al. Methods in Enzymology 465: 21-41 (2009); Drummond et al. Vitamins & Hormones 60: 285-332 (2000), all of which are incorporated herein by reference).

Nanoparticle compositions disclosed herein further include immunoliposomes. Methods for inclusion of an antibody or tumor targeting ligand into the micelle formulation to produce immunoliposomes are known in the art and described further below. For example, methods for preparation and use of immunoliposomes are described in U.S. Pat. Nos. 4,957,735, 5,248,590, 5,464,630, 5,527,528, 5,620,689, 5,618,916. 5,977,861, 6,004,534, 6,027,726, 6,056,973, 6,060,082, 6,316,024, 6,379,699, 6,387,397, 6,511,676 and 6,593,308.

Several types of agents can be encapsulated into the biocompatible vehicle of the nanoparticle composition. For instance, paramagnetic particles can be incorporated into the nanoparticle composition. Paramagnetic particles are composed of compounds that have at least some electrons with unpaired spins. Exemplary paramagnetic particles are magnetite, SPIONs, aluminum, platinum, manganese, and rare earth ions.

Additionally radiolabels can be encapsulated into the nanoparticle composition. Examples of radiolabels include ¹¹¹In, ¹²⁵I, ¹²³I, ¹²⁴I, ¹²⁹I, ¹³¹I, and ⁷⁷Br. The choice of a suitable radioisotope can be optimized based on a variety of factors including the type of radiation emitted, the emission energies, the distance over which energy is deposited, and the physical half-life of the radioisotope.

In some embodiments, fluorophores are encapsulated into the nanoparticle composition. Exemplary fluorophores include fluorescein (FITC), phycoerythrin, and rhodamine. Furthermore, positron emission tomography agents can be included in the nanoparticle composition. Examples of positron emission tomography agents include [F-18]-fluoro-3′-deoxy-3′-L-fluorothymidine (FLT), 2′-fluoro-5-methyl-1-(beta-D-2-arabinofuranosyl)uracil (FMAU), and (F-18) fluorodeoxyglucose (FDG).

In certain embodiments, the nanoparticle composition encapsulates one or more agents. In particular, the nanoparticle compositions can encapsulate two or more agents, three or more agents, and four or more agents. For instance, a cationic liposome can comprise SPION and ¹¹¹In. In other embodiments, a cationic liposome can comprise SPION, ¹¹¹In, and fluorescein (FITC). In still other embodiments, a cationic liposome can comprise SPION, “¹¹¹In, and [F-18 ]-fluoro-3′-deoxy-3′-L-fluorothymidine (FLT).

The nanoparticle compositions can be from about 30 nm to about 250 nm. In certain embodiments, the nanoparticle compositions are from about 50 nm to about 200 nm. In other embodiments, the nanoparticle compositions are from about 75 nm to about 175 nm. In particular embodiments, the nanoparticle compositions are from about 100 nm to about 150 nm.

### 3. Methods of Multimodal Imaging

Aspects disclosed herein include methods of multi-modal diagnostic imaging. The methods involve administering nanoparticle compositions disclosed herein. In particular embodiments, the nanoparticle composition comprises biocompatible vehicles such as liposomes that encapsulate one ore more agents of the group consisting of paramagnetic particles, radiolabels, fluorophores, and positron emission tomography agents. In more particular embodiments, the nanoparticle compositions encapsulate three or more agents. The methods disclosed herein are useful for theranostic procedures for targeting, imaging, and drug and biologic release capabilities.

The methods disclosed herein allow the nanoparticle composition to bind to a tissue. As used herein, “bind” means to associate by any means. Binding is by chemical means and includes covalent binding, van der Waals interactions, magnetic interactions, and ionic interactions. In certain embodiments, the nanoparticle compositions are localized to particular diseased tissues by such binding interactions. In particular embodiments, the nanoparticle compositions are directed to the diseased tissues by antibody interactions with cell-specific biomarkers. Such cell-specific markers are known in the art.

As disclosed herein, nanoparticle compositions can be localized to tissues through magnetophoretic means disclosed herein. Briefly, nanoparticle compositions comprising paramagnetic particles are injected into a subject. Once the paramagnetic particles are injected, magnetic targeting can be achieved using a magnet, such as a 1 cm², 0.4 T magnet, attached over the area of interest using surgical tape. The magnet is left in place during the first hour post-injection to guide nanoparticle compositions containing paramagnetic particles to the diseased tissues. In particular instances, diseased tissues are tumors.

In certain aspects, the nanoparticle compositions are administered such that they circulate in the vasculature in the subject. The nanoparticle compositions can bind to, or localize to, the endothelium of the blood vessels of the vasculature. In these instances, the blood vessels can be imaged in the same way as other tissues.

The methods further entail detecting the nanoparticle composition by one or more imaging techniques selected from the group consisting of positron emission tomography (PET), magnetic resonance imaging (MRI), single photon emission computed tomography (SPECT/CT), and optical imaging. Other non-limiting examples of medical imaging systems include (a) X-ray based Computer Tomography (CT), and combinations and improvements on these technologies [(PET+CT, spiral CT, single photon emission CT (SPECT), high resolution PET (microPET), and immunoscintigraphy (using radiolabeled antibodies (Czernin, J. and M. E. Phelps, Ann. Rev. Med. 53:89-112 (2002); Goldenberg, D. M., Cancer 80 (12):2431-2435 (1997); Langer, S. G. et al. World J. Surg. 25:1428-1437 (2001); Middleton M L, Shell E G., Postgrad Med. 11l(5):89-90, 93-6 (2002); (b) magnetic resonance imaging (MRI) (Helbich, T. H., J. Radiol. 34:208-219, (2000); Langer, S. G. et al. World Journal of Surgery 25:1428-1437 (2001); Nabi, H. A. and Zubeldia, J. M., Oncology J. Nuclear Med. Technol. 30 (1):3-9 (2002); ultrasonic imaging (US) (Harvey, C. J., et al. Advances in Ultrasound Clin. Radiol. 57:157-177 (2002); Langer, S. G. et al. W. J. Surg. 25:1428-1437 (2001)); (c) fiber optic endoscope (Shelhase D E, Curr. Opin. Pediatr. 14:327-33 (2002)); (d) gamma scintillation detectors (detect gamma emitters, e.g. 192-Ir), and beta scintillation detectors (detect beta emitters, e.g. 90-Sr/Y) (Hanefeld C, Amirie, S. et al., Circulation 105:2493-6 (2002)).

The methodologies also include generating one or more images of the tissue bound by the nanoparticle composition or of the circulation system and registering the images from different modalities to obtain an accurate location of the tissue and or the organs being imaged. Such techniques allow for highly accurate localization of tissues and, in particular, diseased tissues within a subject. In addition, the techniques allow for specific localization of diseased cells within healthy tissue.

### 4. Methods of Treating Disease

The methods disclosed herein provide for treatment of diseases, such as cancer. The cancers treated by the disclosed methods including, but not limited to, neoplasms, tumors, metastases, or any disease or disorder characterized by uncontrolled cell growth. Examples of types of cancer and proliferative disorders to be treated include, but are not limited to, leukemia (e.g., myeloblastic, promyelocytic, myelomonocytic, monocytic, erythroleukemia, chronic myelocytic (granulocytic) leukemia, and chronic lymphocytic leukemia), lymphoma (e.g., Hodgkin's disease and non-Hodgkin's disease), fibrosarcoma, myxosarcoma, liposarcoma, chondrosarcoma, osteogenic sarcoma, angiosarcoma, endotheliosarcoma, Ewing's tumor, colon carcinoma, pancreatic cancer, breast cancer, ovarian cancer, prostate cancer, squamous cell carcinoma, basal cell carcinoma, adenocarcinoma, renal cell carcinoma, hepatoma, Wilms' tumor, cervical cancer, uterine cancer, testicular tumor, lung carcinoma, small cell lung carcinoma, bladder carcinoma, epithelial carcinoma, glioma, astrocytoma, oligodendroglioma, melanoma, neuroblastoma, retinoblastoma, dysplasia and hyperplasia. In a particular embodiment, therapeutic compounds of the invention are administered to men with prostate cancer (e.g., prostatitis, benign prostatic hypertrophy, benign prostatic hyperplasia (BPH), prostatic paraganglioma, prostate adenocarcinoma, prostatic intraepithelial neoplasia, prostato-rectal fistulas, and atypical prostatic stromal lesions). The treatment of cancer includes, but is not limited to, alleviating symptoms associated with cancer, the inhibition of the progression of cancer, the promotion of the regression of cancer, and the promotion of the immune response.

The methods disclosed herein utilize the disclosed nanoparticle compositions to treat diseased tissues. The nanoparticle compositions are administered to subjects. The nanoparticle compositions can perform two functions. The first is to image the diseased tissues to which the nanoparticle compositions are localized. The second function is to eliminate the diseased tissues or cells to which the nanoparticle compositions have either localized or bound. The nanoparticle composition is administered and comprises two or more of the group consisting of paramagnetic particles, radiolabels, fluorophores, and positron emission tomography agents.

Certain methods utilize magnetic fields to generate hyperthermia to treat diseased tissues. In such embodiments, the nanoparticle composition is subjected to a magnetic field such that the temperature of the nanoparticle composition increases; thereby killing the diseased tissue. For such therapy, ac magnetic fields are generated at frequencies between 1 kHz to 1 MHz. In addition, the amplitude of the ac current can be 50-500 Oe or 5-50 mTesla. The diseased tissues (e.g., solid tumors) are exposed to high temperatures for a sustained period of time. Local hyperthermia at temperatures up to 45° C. enhances the killing of tumor cells exposed to chemotherapeutics by increasing the susceptibility to ionizing radiation and promoting better perfusion of systemically-administered drugs into the tumor mass. It can also lead to decrease in functional P-glycoprotein efflux pump leading to effective cell kill. Thus localized hyperthermia used as an adjuvant to chemotherapy can overcome multi-drug resistance (MDR) and in combination with radiation therapy can overcome radiation resistance. Thermal ablation therapy typically at temperatures exceeding 60° C. can be used as a minimally invasive knife-less alternative to surgical resection of tumors.

In certain embodiments, a magnetic nanoliposome is used. The liposome encapsulates SPION in its aqueous core, as well as fluorophores and radiolabels as imaging agents. The liposomes, in these embodiments, are a single nanoplatform that have all-in-one multifunctional capability for targeting through its cationicity and magnetic guidance using an external dc field to accumulate in the tumor, imaging using MRI, SPECT and optical fluorescence, and absorbs energy from applied oscillating magnetic fields to achieve hyperthermia, ablation and necrosis.

Magnetic nanoparticles when subjected to magnetic field strengths of certain frequency can result in heating. The physical basis of this heating in particles suspended in liquid by AC magnetic field is very well understood. The critical size for this phenomenon at around 300 kHz is about 20 nm. Each NP can be viewed as a small magnet with a magnetic moment in and around this size; the energy absorption is mainly due to the relaxation of these superparamagnetic NPs as a response to the external alternating magnetic field. For NPs, with an anisotropy energy, ^{K}, having KV>>kT where ^{V} is the particle volume, the specific power loss due to Neel Relaxation in an alternating current magnetic field H cos(ωt), is given by P=(mHωτ)²/[2τkTV(1+ω²τ²)]. τ=τ₀exp(KV/kT) is the relaxation time of the particle with τ₀=10⁻⁰sec s. The specific power loss depends strongly on the particle radius r=(3V/4π)^{1/3} and frequency of the AC field. Thus depending on the optimum frequency being investigated, the correct particle size has to be selected accordingly. The use of magnetic NPs, in the range of 10-20 nm, offers an effective method of preferential heating using electromagnetic radiation in the frequency range of 50-300 kHz.

Certain aspects disclose methods of treating diseased tissue comprising administering a nanoparticle composition comprising a biocompatible vehicle encapsulating paramagnetic particles and siRNA molecules. The siRNA molecules are released into the cells of the diseased tissue (i.e., into the diseased cells) so that the diseased tissue is treated with the siRNA.

Several siRNA molecules are within the scope of the methods disclosed herein. For example, kinesin spindle proteins (“KSP”) siRNA can be used. An exemplary siRNA sequence for KSP siRNA is CUGAAGACCUGAAGACAAUdTdT (SEQ ID NO: 1). Other exemplary KSP sequences include AUUGUCUUCAGGUCUUCAGdTdT, CUGAAGACCUGAAGACAAUdTdT, CUGAAGACCUGAAGACAAUdTdT, AUUGUCUUCAGGUCUUCAGdTdT, AUUGUCUUCAGGUCUUCAGdTdT. Other exemplary siRNAs include GenBank Accession Nos. NM_{—}05030, NM_{—}001790, NM_{—}001130829, NM_{—}001111045, NM_{—}000321, NM_{—}053056, NM_{—}001114735, NM_{—}001130845, and NM_{—}005178. Additional siRNA sequences useful in the present invention are disclosed at Labome.com and available commercially from Ambion Corp. (Austin, Tex.). Furthermore, siRNA sequences can be ordered from Integrated DNA Technologies (Coralville, Iowa). Additionally, techniques for preparing siRNA molecules is well known in the art (see, e.g., Silencer® siRNA Construction Kit, Ambion Corp., Austin, Tex.).

Doses of siRNA delivered to a subject can be from 1 mg of siRNA/kg subject body mass to 10 mg of siRNA/kg subject body mass. Other ranges include 2 mg/kg to 5 mg/kg.

The MNL disclosed herein can be administered in combination with other therapies (e.g., antibiotics, cancer drugs, antivirals). In addition, MNL can be formulated to encapsulate said therapies with paramagnetic particles and other agents disclosed herein.

### 5. Therapeutic Formulations

The MNL can be provided in therapeutic formulations. The MNL can be administered alone or in combination with other types of cancer treatments (e.g., radiation therapy, chemotherapy, hormonal therapy, immunotherapy and anti-tumor agents). Examples of anti-tumor agents include, but are not limited to, cisplatin, ifosfamide, paclitaxel, taxanes, topoisomerase I inhibitors (e.g., CPT-11, topotecan, 9-AC, and GG-211), gemcitabine, vinorelbine, oxaliplatin, 5-fluorouracil (5-FU), leucovorin, vinorelbine, temodal, and taxol. In one embodiment, the therapies disclosed herein are administered to a subject, such as a mammal (e.g., a human). In another embodiment, the MNL and combination therapies are provided for a period of time (e.g., 1 minute, 15 minutes, 30 minutes, 45 minutes, 1 hour, 2 hours, 4 hours, 6 hours, 8 hours, 12 hours, 24 hours, 2 days, or 1 week before), or for a period of time subsequent to (e.g., 1 minute, 15 minutes, 30 minutes, 45 minutes, 1 hour, 2 hours, 4 hours, 6 hours, 8 hours, 12 hours, 24 hours, 2 days, or 1 week after) radiation therapy or other chemotherapy.

The MNL therapies can be provided in combination with certain antibody therapies. Examples of such antibody therapies include, but are not limited to, Herceptin, Retuxan, OvaRex, Panorex, BEC2, IMC-C225, Vitaxin, Campath I/H, Smart MI95, LymphoCide, Smart I D10, and Oncolym.

Further provided are methods for the treatment of viral and other pathogen infections in a subject, the methods comprising the administration of a therapeutically or prophylactically effective amount of MNL or combination therapies. Examples of viral infections which can be treated or prevented in accordance with this invention include, but are limited to, viral infections caused by retroviruses (e.g., human T-cell lymphotrophic virus (HTLV) types I and II and human immunodeficiency virus (HIV)), herpes viruses (e.g., herpes simplex virus (HSV) types I and II, Epstein-Barr virus and cytomegalovirus), arenaviruses (e.g., lassa fever virus), paramyxoviruses (e.g., morbillivirus virus, human respiratory syncytial virus, and pneumovirus), adenoviruses, bunyaviruses (e.g., hantavirus), cornaviruses, filoviruses (e.g., Ebola virus), flaviviruses (e.g., hepatitis C virus (HCV), yellow fever virus, and Japanese encephalitis virus), hepadnaviruses (e.g., hepatitis B viruses (HBV)), orthomyoviruses (e.g., Sendai virus and influenza viruses A, B and C), papovaviruses (e.g., papillomavirues), picomaviruses (e.g., rhinoviruses, enteroviruses and hepatitis A viruses), poxviruses, reoviruses rotavirues), togaviruses (e.g., rubella virus), and rhabdoviruses (e.g., rabies virus). The treatment of a viral infection includes, but is not limited to, alleviating symptoms associated with said infection, the inhibition or suppression of viral replication, and the enhancement of the immune response.

The MNL described herein can be administered alone or in combination with other types of anti-viral or other anti-pathogen agents. Examples of anti-viral agents include, but are not limited to: cytokines (e.g., IFN-.alpha., IFN-.beta., and IFN-.gamma.); inhibitors of reverse transcriptase (e.g., AZT, 3TC, D4T, ddC, ddI, d4T, 3TC, adefovir, efavirenz, delavirdine, nevirapine, abacavir, and other dideoxynucleosides or dideoxyfluoronucleosides); inhibitors of viral mRNA capping, such as ribavirin; inhibitors of proteases such HIV protease inhibitors (e.g., amprenavir, indinavir, nelfinavir, ritonavir, and saquinavir,); amphotericin B; castanospermine as an inhibitor of glycoprotein processing; inhibitors of neuraminidase such as influenza virus neuraminidase inhibitors (e.g., zanamivir and oseltamivir); topoisomerase I inhibitors (e.g., camptothecins and analogs thereof); amantadine and rimantadine.

In certain embodiments, hemotherapeutic agent such as Actinomycin, Adriamycin, Altretamine, Asparaginase, Bleomycin, Busulfan, Capecitabine, Carboplatin, Carmustine, Chlorambucil, Cisplatin, Cladribine, Cyclophosphamide, Cytarabine, Dacarbazine, Dactinomycin, Daunorubicin, Docetaxel, Doxorubicin, Epoetin, Etoposide, Fludarabine, Fluorouracil, Gemcitabine, Hydroxyurea, Idarubicin, Ifosfamide, Imatinib, Irinotecan, Lomustine, Mechlorethamine, Melphalan, Mercaptopurine, Methotrexate, Mitomycin, Mitotane, Mitoxantrone, Paclitaxel, Pentostatin, Procarbazine, Taxol, Teniposide, Topotecan, Vinblastine, Vincristine, or Vinorelbine. In particular embodiments, the therapeutic component is in a liposome formulation.

Those skilled in the art will recognize, or be able to ascertain, using no more than routine experimentation, numerous equivalents to the specific substances and procedures described herein. Such equivalents are intended to be encompassed in the scope of the claims that follow the examples below.

### EXAMPLES

### Example 1

Magnetic cationic liposomes are positively-charged vesicles containing fluid MAG-C (magnetite). MAG-C attracts cationic liposomes to tumor microvasculature.

The following experiments investigated liposome size, zeta potential (surface charge potential), and phase transition temperature of potentially useful preparations.

### 1. Exemplary Liposomal Components

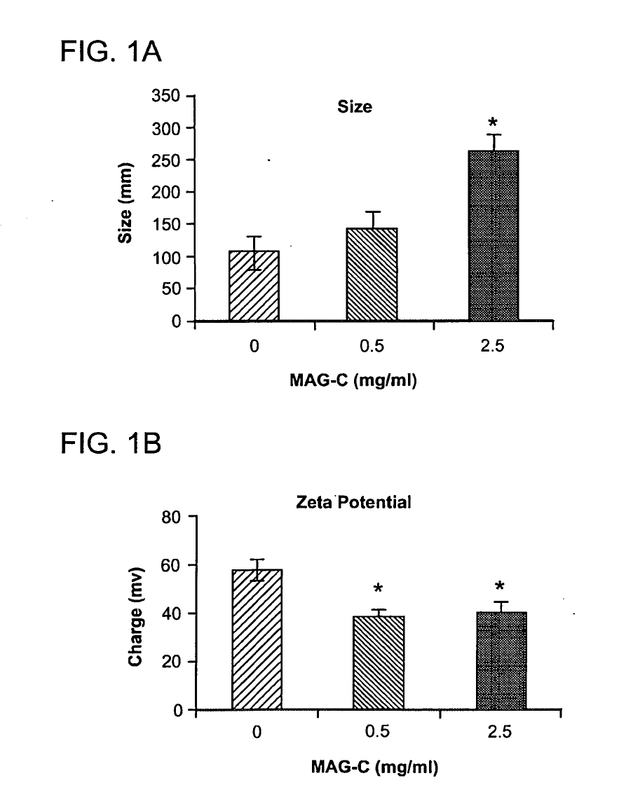

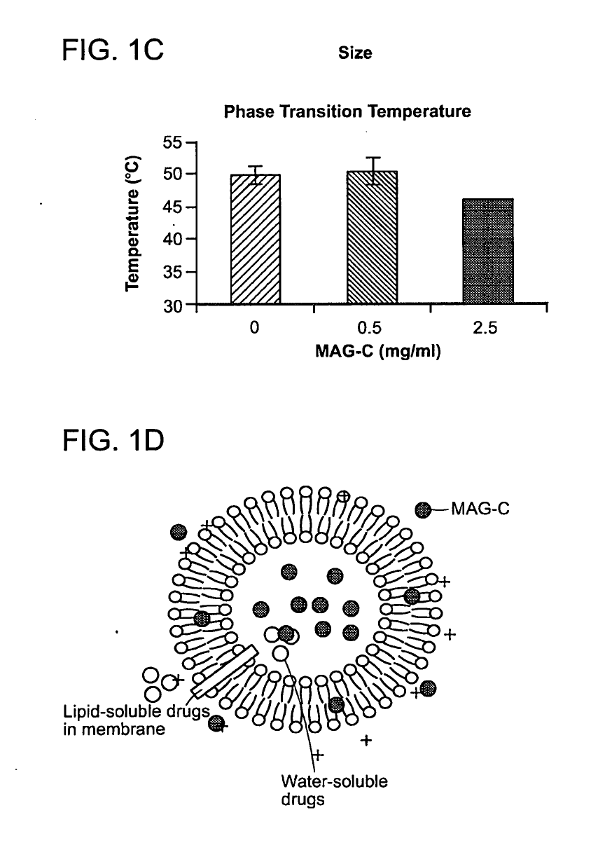

Several ingredients were used to prepare MAG-C cationic liposomes, an pictorial representation of which is shown in FIG. 1D. The extent to which each component exerted its effect on bilayer physical properties was determined (see Tables 1 and 2).

TABLE 1
Preparation of liposomal formulations

| Group | Liposome preparation | Lipid ratio (mol %) |
|---|---|---|
| 1 | DMPC | 100 |
| 2 | DMPC/DMTAP | 50:50 |
| 3 | DMPC/DMTAP/CHOL | 40:50:10 |
| 4 | DMPC/DMTAP/CHOL/PEG | 35:50:10:5 |
| 5 | DMPC/DMTAP/CHOL/MAG-C | 40:50:10 |
| 6 | DMPC/DMTAP/CHOL/MAG-C/PEG | 35:50:10:5 |

TABLE 2
Physical characterization of liposomes: the size and zeta potential values for
different liposomal preparations were determined immediately following 10 min of sonication

| | | | | Group | P values | |
|---|---|---|---|---|---|---|
| Group | Liposome preparation | Size (nm) | Zeta potential (mv) | comparisons | Size | Zeta potential |
| 1 | DMPC | 483 ± 187.02 | −0.26 ± 3.99 | — | — | — |
| 2 | DMPC/DMTAP | 369 ± 144.38 | 84.96 ± 15.57 | 1 and 2 | NS | P < 0.05 |
| 3 | DMPC/DMTAP/CHOL | 105 ± 26.64 | 64.55 ± 16.68 | 2 and 3 | P < 0.01 | NS |
| 4 | DMPC/DMTAP/CHOL/PEG | 109 ± 5.31 | 26.24 ± 2.28 | 3 and 4 | NS | P < 0.01 |
| 5 | DMPC/DMTAP/CHOL/MAG-C (0.5 mg/ml) | 142 ± 27.40 | 43.79 ± 10.93 | 3 and 5 | NS | P = 0.05 |
| 6 | DMPC/DMTAP/CHOL/MAG-C/PEG | 141 ± 14.52 | 21.98 ± 5.25 | 5 and 6 | NS | P < 0.01 |
| 7 | DMPC/DMTAP/CHOL/MAG-C (2.5 mg/ml) | 267 ± 27.43 | 39.82 ± 5.26 | 3 and 7; 5 and 7 | P < 0.05; P < 0.05 | P < 0.05 NS |
| 8 | DMPC/DMTAP/CHOL/MAG-C/PEG | 156 ± 16.25 | 27.56 ± 3.54 | 7 and 8 | P < 0.05 | P < 0.05 |

MAG-C containing liposome were centrifuged at 1000x g for 15 min to remove unincorporated MAG-C.

### 2. Liposome Size

Changes in liposome size and dispersity are important indicators of change in bilayer properties of liposomes. The following values reported for liposome size were determined following 10 min of sonication.

As shown in FIG. 1A, the size of DMPC liposomes (483 nm±187.02 nm) tended toward smaller mean diameters when the cationic lipid DMTAP was included as a component of the preparation. We also investigated the effect of including cholesterol in DMPC/DMTAP preparations; cholesterol significantly reduced liposome diameter of DMPC/DMTAP liposomes from 369 nm±144.38 nm to 105 nm±26.64 nm. When MAG-C (magnetite) was added at 0.5 mg/ml there was no significant change in mean diameter of DMPC/DMTAP/cholesterol liposomes, but higher MAG-C content (2.5 mg/ml) resulted in a significant increase in mean diameter from 105 nm±26.64 nm to 267 nm±27.43 nm.

### 3. Zeta Potential

Quantitative changes in surface charge characteristics of liposomes due to the inclusion of different liposome components can be used to predict the relative affinity of tumor vessels for liposomes on the basis of charge. Zeta potential values were determined immediately following size measurements, and were therefore the identical preparations used for the determination of size (FIG. 1B).

Zeta potential for electroneutral liposomes consisting of DMPC alone was −0.26 mv±3.99 mv. This value increased significantly in the presence of 50 mol % DMTAP (84.96 mv±15.57 mv; P<0.05). The inclusion of 10 mol % cholesterol in DMPC/DMTAP preparations reduced zeta potential from 84.96 mv±15.57 mv to 64.55 mv±16.68 mv (P>0.05). The inclusion of 0.5 mg/ml of MAG-C into cationic liposomes significantly reduced the zeta potential of MAG-C cationic liposomes (43.79 mv±10.93 mv) compared to preparations without MAG-C (64.55 mv±16.68 mv). An increase inMAG-C (2.5 mg/ml) content resulted in no significant changes in zeta potential compared to preparations containing 0.5 mg/ml of MAG-C (FIG. 1B).

### 4. Membrane Fluidity

DPH (diphenylhexatriene), a fluorescent membrane probe, was used to determine specific changes in membrane fluidity due to components added to the liposome preparation. In liposomes of DMPC alone, DPH had a polarization value of approximately 0.31 at 12° C. and a phase transition temperature of 26° C. The inclusion of DMTAP (50 mol %) significantly increased the polarization values to 0.45 at 12° C. The phase transition temperature for liposomes of DMPC/DMTAP was 42° C. DMTAP therefore reduced the fraction of liquid crystalline phase compared to DMPC alone, as observed between temperatures of 12° C. to 45° C. After 45° C., DMPC and DMPC/DMTAP liposomes had similar polarization values and existed predominately in the liquid crystalline phase state (FIG. 1C). Incorporation of cholesterol (10 mol %) increased the phase transition temperature of DMPC/DMTAP liposomes from 42° C. to 50° C. The bilayer properties become more rigid in the presence of cholesterol compared to liposomes of DMPC/DMTAP alone.

Thus, higher temperatures were required to melt the lipid bilayer in the presence of cholesterol. The phase transition temperature of cationic liposomes was not altered by the inclusion of low concentrations of MAG-C (0.5 mg/ml) (FIG. 1C). As the MAG-C concentration was increased to 2.5 mg/ml the phase transition temperature decreased from 50° C. to 46° C. (FIG. 1C).

### 5. Cell Viability Assay

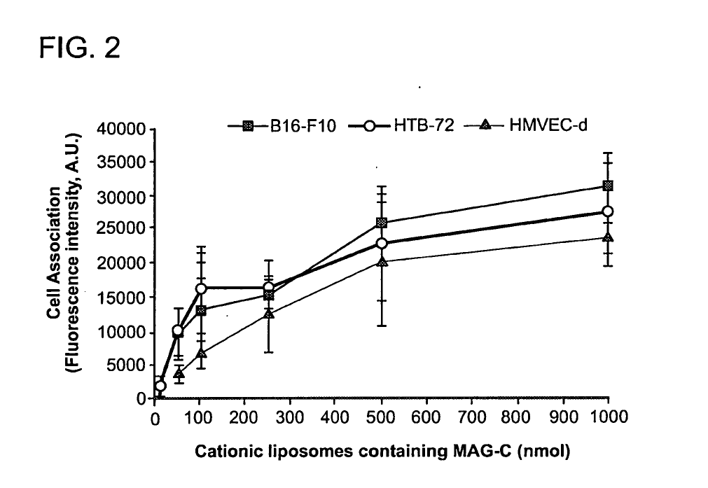

We observed no toxic effects on cell growth against murine melanoma (B16-F10), human melanoma (HTB-72), and human dermal microvascular endothelial (HMVEC-d) cells at <1000 nmol (FIG. 2). The percent of viable B16-F10, HTB-72, and HMVEC-d cells after 24 h exposure to 500 nmol of liposomes was 92%, 85%, and 78%, respectively. Although the values reported here for HMVEC-d tended toward relatively lower cell viabilities, no statistically significant effects were observed. The percent of viable cells remaining in culture after exposure to MAG-C cationic liposomes reduced significantly at ≧500 nmol compared to 10 nmol with all three cell lines. Moreover, we observed no significant difference in cell viability among cell lines at any given concentration, suggesting that the liposomes were equally toxic to both cancer and microvascular endothelial cells.

### 6. Cell Association Studies

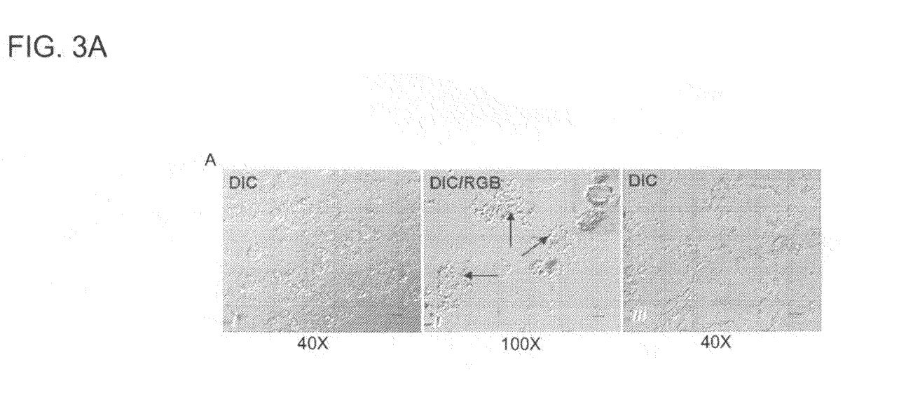

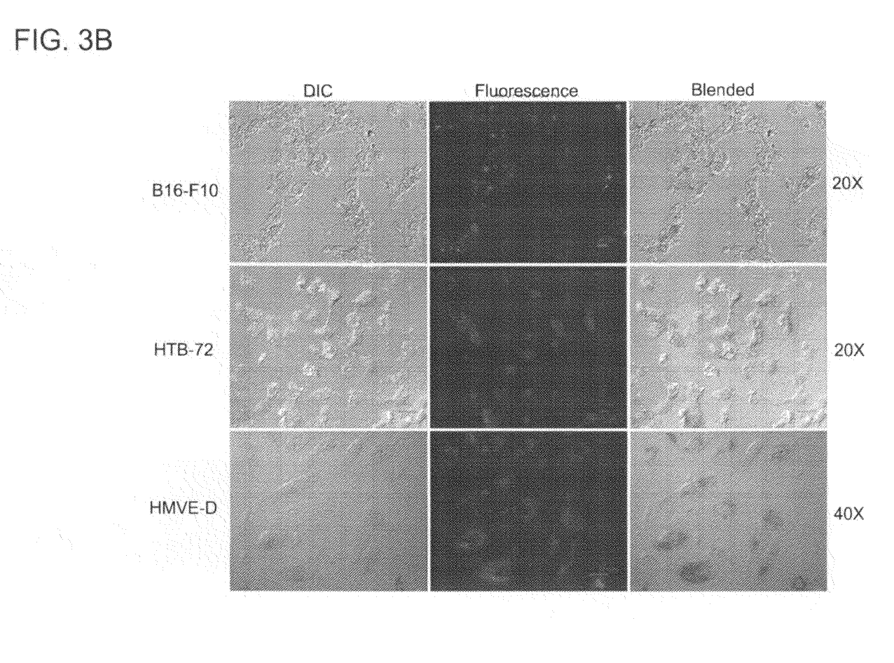

The tendency of MAG-C cationic liposomes to associate with cells increased with liposome amount, and association values were similar for the three cell lines. We observed no significant increase in cell association >500 nmol of liposomes (FIGS. 3A-3B). DIC and fluorescence microscopic imaging show that avid accumulation of MAG-C cationic liposomes was evident in all cell lines with the most significant uptake observed in B16-F10 and HTB-72. In addition, we note perinuclear uptake in HMVEC-d cells.

### 7. Role of PEG in MAG-C Cationic Liposomes

A sterically stabilized liposome is one that is surface coated with polyethylene glycol (PEG), or some other polymer (Torchilin, et al. (1995) Adv. Drug Del. Rev. 16:141-155). One purpose of including PEG in preparations is to limit the interaction of liposomes with opsonins in blood; acquisition to proteins in blood rapidly eliminates them from systemic circulation (Ishida, et al. (2002) Biosci. Rep. 22:197-224). Liposomes bearing a high cationic surface charge potential are the most sensitive to this mechanism of elimination. Prolonging the circulation half-life of MAG-C cationic liposomes increases overall tumor vascular targeting efficiency due to extended access to the external magnetic field. We therefore investigated the effect of PEG on bilayer physical properties. We determined the effect of 5 mol % DMPE-PEG on size, and zeta potential, of cationic liposomes containing MAG-C. The following studies compared MAG-C cationic liposomes to MAG-C PEGylated cationic liposomes (MAG-C PCLs). The size of MAG-C PCLs (containing 0.5 mg/ml) was 142.56±14.22 and was similar to values reported for MAG-C liposomes in absence of PEG (141.143±31.55). PEG reduced zeta potential of cationic liposomes containing MAG-C, showing a value of 21.98 mv±5.25 my compared to preparations without PEG (43.79 mv±10.93 mv). The inclusion of DMPE-PEG to the cationic liposome preparation containing relatively high MAG-C (2.5 mg/ml) content resulted in a significant decrease in liposome size from 262.67 nm±27.43 nm to 156 nm±nm 16.25 nm.

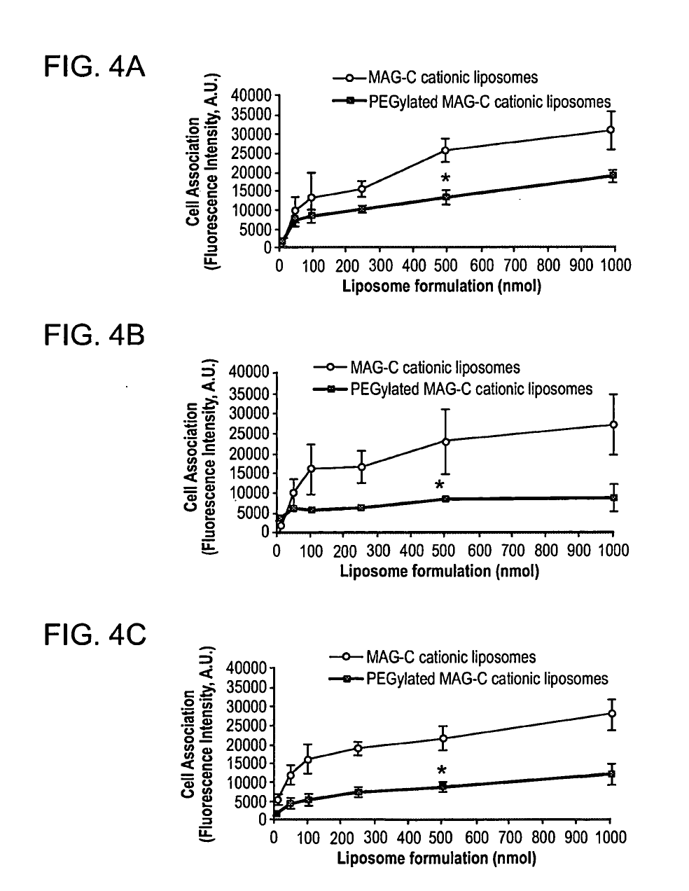

We next compared the interaction of PCLs with cancer (B16-F10 and HTB-72) and endothelial (HUVEC) cell lines (FIGS. 4A-4C).

We observed that the inclusion of PEG significantly reduced cellular interactions regardless of cell type (FIGS. 4A-4C). These data suggest that the inclusion of 5 mol % PEG altered bilayer physical properties such as liposome cationic surface charge potential, but did not affect liposome size. PEG limited the association of MAG-C cationic liposomes with all cell lines. Such reduced interactions may be due to the reported decrease in zeta potential (P<0.01).

### 8. In Vivo Tissue Distribution Profile of 111 In-Labeled MAG-C Cationic and PEGylated MAG-C Cationic Liposomes

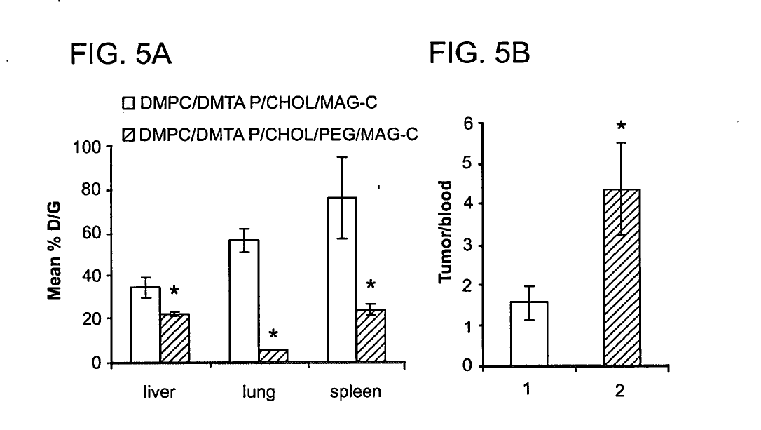

We next determined the effect of PEG on tissue distribution profile of MAG-C cationic liposomes in melanoma bearing SCID mice. When MAG-C cationic liposomes were injected intravenously into melanoma bearing mice, the percent of injected dose per gram of the tissue in lung, liver and spleen was 35.27%, 56.61%, and 75.92%, respectively (FIGS. 5A-5B). The inclusion of 5 mol % DMPE-PEG resulted in a significant reduction in the uptake by the major organs of RES. The significant reduction was due to the electrostatic repulsive effect of PEG (Ishida, et al. (2002) Biosci. Rep. 22, 197-224; Torchilin, et al. (1995) Adv. Drug Del. Rev. 16, 141-155). Moreover, tumor/blood ratio for PEGylated MAG-C cationic liposomes (4.36±1.14) was significantly higher than that for MAG-C cationic liposomes (1.56±0.43). Thus, PEG minimizes uptake of MAG-C cationic liposomes by healthy tissues while improving tumor uptake.

### 9. Iron Content

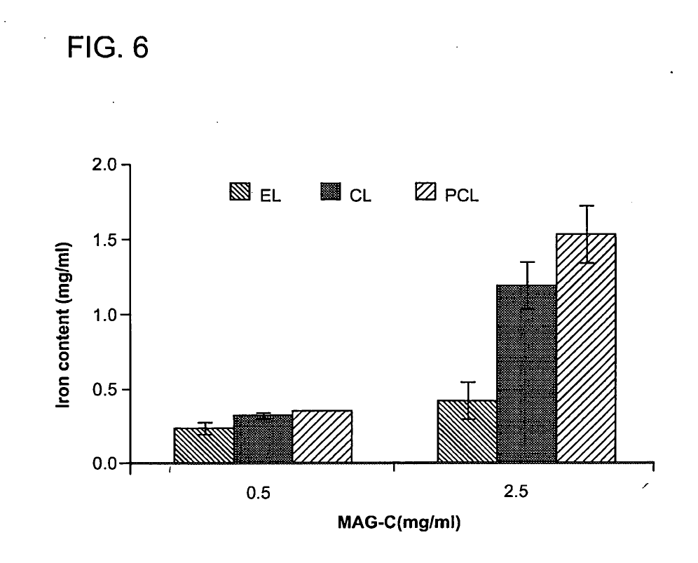

The fraction of magnetite loaded must be sufficient to respond to an external magnetic field (Alexiou, et al. (2000) Can. Res. 60(23):6641-8; Babincova, et al. (2000), Z. Naturforsch [C], 55(3-4):278-81). Therefore, the amount of iron incorporated in each liposome variety was determined spectrophotometrically. The iron content in MAG-C is 72%. So 0.5, 2.5 mg/ml of MAG-C corresponds to 0.36 and 1.8 mg/ml of iron, respectively. When MAG-C was added at 0.5 mg/ml (iron content of −0.36 mg/ml) into electroneutral liposomes (EL), cationic liposomes (CL) and PEGylated cationic liposomes (PCL), the percent of iron incorporated was significantly higher for CL (89%) and PCL (93%) compared to EL (64%). The corresponding specific amount of iron incorporated in EL, CL, and PCL was 0.2370.04 mg/ml, 0.3270.01 mg/ml, and 0.34 mg/ml, respectively (FIG. 6). In general, an increase in MAG-C from 0.5 mg/ml to 2.5 mg/ml (iron content of −1.8 mg/ml) influenced the percent of iron loaded in liposomes however, the liposome composition appeared to determine the percent and quantitative amount of iron incorporated. For example, cationic liposomes incorporated significantly higher MAGC (65%) compared to electroneutral liposomes (23%), and the inclusion of DMPE-PEG5000 (5 mol %) increased loading from 65% to 85%. Starting with 1.8 mg/ml of iron content (from the 2.5 mg/ml of MAG-C liposome preparations), the amount of iron incorporated in EL, CL, and PCL was 0.4170.12 mg/ml, 1.1770.17 mg/ml, and 1.5370.2 mg/ml, respectively. TEM images confirmed association of MAG-C with cationic liposomes. Moreover, TEM showed relatively high association of MAG-C with cationic liposomes containing 2.5 mg/ml compared to 0.5 mg/ml.

10. Drug Loading into MAG-C Cationic Liposomes

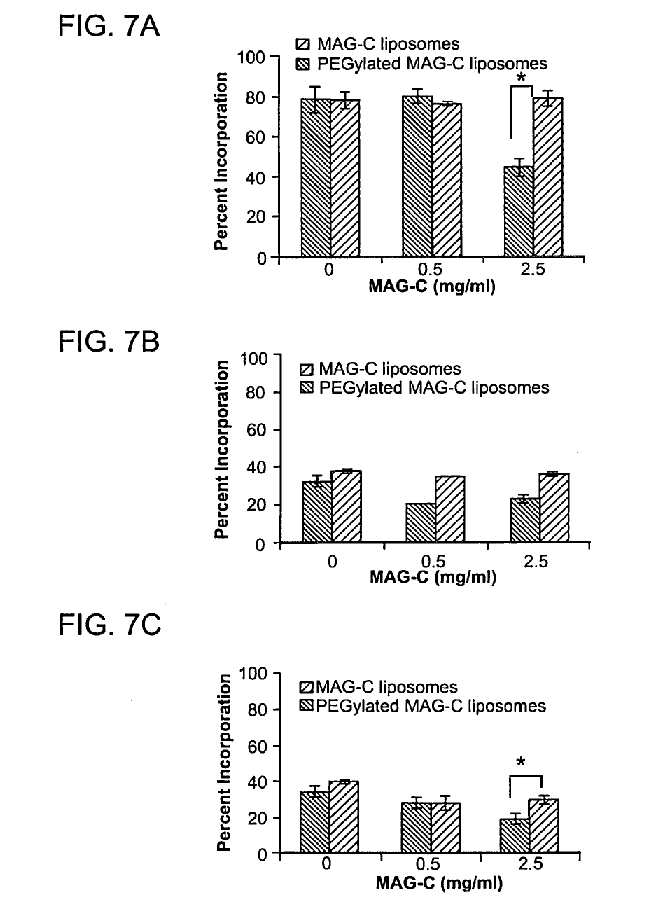

The influence of MAG-C on the efficiency of loading chemotherapeutic agents (hydrophilic and lipophilic) in cationic liposomes was evaluated by HPLC. Inclusion of MAG-C at lower concentrations (0.5 mg/ml) did not alter the incorporation efficiency of lipophilic drug etoposide (3 mol %), but at higher concentrations (2.5 mg/ml) the incorporation efficiency decreased from 7876.03% to 4474.26% (FIGS. 7A-7C). When PEG was included as component of MAG-C cationic liposomes the efficiency of drug loading increased from 4474.26% to 7873.73%. For the water soluble drug vinblastine sulfate (3 mol %) loading efficiency decreased from 3273.6% to 21.1570.11% with 0.5 mg/ml of MAG-C but no further decrease was observed with higher MAG-C content (2.5 mg/ml). The inclusion of PEG in MAG-C cationic liposomes improved incorporation of vinblastine sulfate from 2170.11% to 3570.01%. We also evaluated the effect of MAG-C on the incorporation efficiency of the hydrophilic drug, dacarbazine at 3 mol %. The addition of MAG-C at lower concentrations (0.5 mg/ml) did not influence the incorporation efficiency of dacarbazine (2873.02%); efficiency of loading however decreased from 2873.02% to 1973.2% with 2.5 mg/ml MAG-C. As observed with etoposide and vinblastine sulfate, PEG significantly improved the incorporation of dacarbazine in cationic liposomes from 1973.2% to 3071.58%.

### 11. Measurement of Magnetic Susceptibility

We investigated magnetic susceptibility of magnetic cationic liposomes.

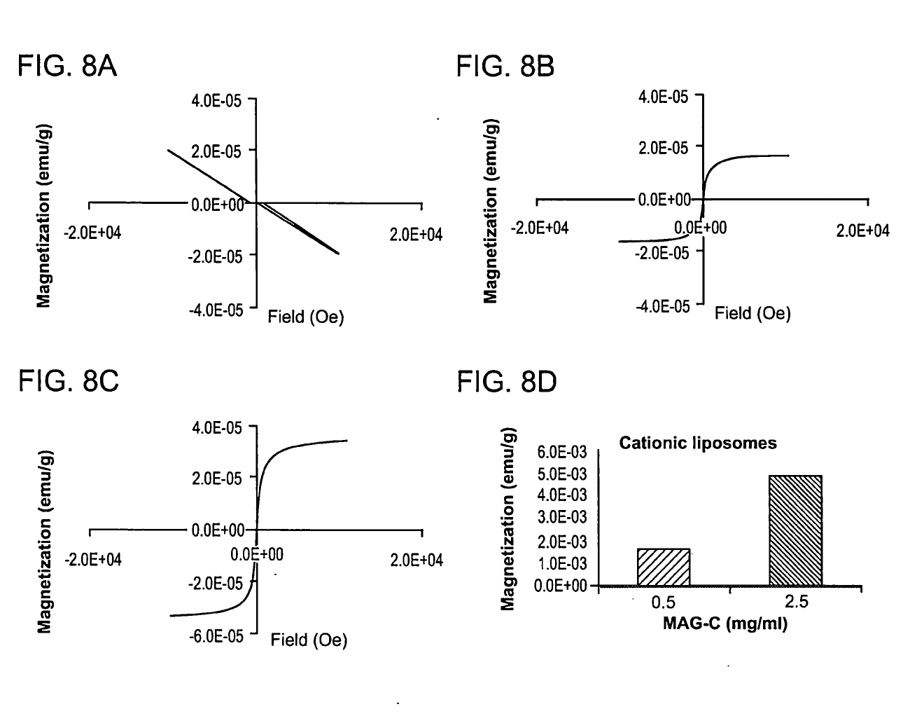

FIGS. 8A-8D display the magnetization curve as a function of the magnetic field for each preparation type. Cationic liposomes (without MAG-C) showed the typical curve for diamagnetic materials confirming a lack of magnetic susceptibility. The magnetization curves of MAG-C cationic liposomes did not show a hysteresis loop confirming the superparamagnetic behavior of magnetite. The saturation magnetization value of magnetic cationic liposomes containing 2.5 mg/ml of MAG-C (4.8_{—}10_{—}3 emu/g of lyophilized liposome) was significantly higher than 0.5 mg/ml (1.6_{—}10_{—}3 emu/g of lyophilized liposome), The values for magnetic susceptibility correlated with the quantity of iron measured in each preparation.

### 12. Tumor Accumulation of MAG-C Cationic Liposomes in Presence of External Magnet

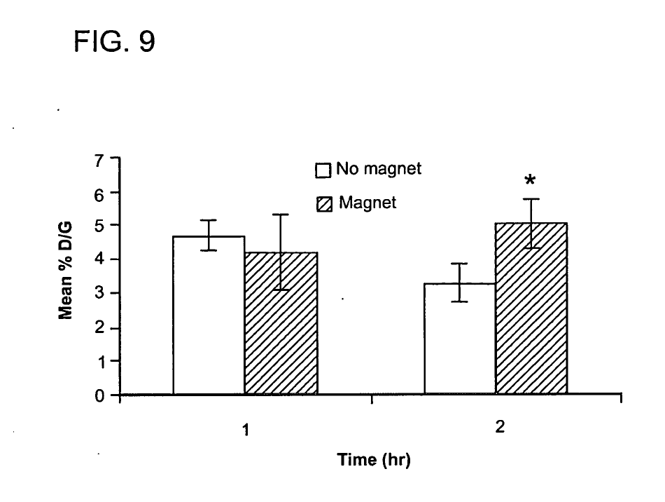

We have examined the effect of the external magnet (1.2 T) on biodistribution of MAG-C cationic liposomes in B16-F10 melanoma bearing mice. First, the external magnet was placed on the external surface of the tumor followed by systemic injection of 111In labeled MAG-C cationic liposomes. The magnet remained associated with the tumor mass for 1 h. The percent of label recovered by the tumor following exposure to external magnet was compared to the (unexposed) control group. At the 1 h time point we observed no significant difference between the two groups, as determined by the similar percent of label recovered by tumors in the presence (4.2±1.08) and absence (4.71±0.4) of magnet (FIG. 9). We however, did observe a significant difference at the 2 h time point (or 1 h following the removal of magnet). At this time interval tumors with no previous exposure to external magnet retained significantly less of the label, compared to tumors previously exposed to magnet (P <0.05). Percent of label recovered by tumor in absence and presence of external magnet at 2 h time point was (5.04±0.73) and (3.3±0.54), respectively. The experimental findings suggest that the external magnet significantly enhanced tumor retention and facilitated vascular uptake of MAG-C cationic liposomes. Enhanced tumor vascular association is the preferred mechanism of uptake given the natural affinity of cationic liposomes for tumor vessels (Thurston, et al. (1998) J. Clin. Invest. 101, 1401-1413; Campbell, et al. (2002) Can. Res. 62, 6831-6836; McLean, et al. (1997) Am J. Physiol., 273, H387-H404).

13. MRI Imaging with MCL

MR imaging was performed on a 7 T preclinical MRI system (BioSpec 70/20USR, Bruker BioSpin Corp., Billerica, Mass.) at the Center for Translational Neuro-Imaging at Northeastern University. A whole body quadrature coil was used for reception and emission.

A. Healthy Mice

A study of 10-week-old healthy SCID mice was carried out to determine the best imaging sequence and parameters to obtain optimal contrast from and get acquainted with the magnet. T2-weighted Turbo-RARE (TR[msec]/TE[msec]=4200/12), T1-weighted RARE spin-echo (1738/10), and T2*-weighted gradient-echo (722.7/4.9, 30° flip angle) were performed with coronal slices 1 mm thick covering the whole animal. Animals were imaged before and immediately after injection of 100 μL of a 5 mg/mL of magnetite encapsulated in cationic liposomes (about 200 nm). These initial images gave great insight as to where magnetic cationic liposomes (MCL) accumulate and whether or not the method was successful in labeling the liposome formulation by providing negative contrast on MR images.

The study on healthy animals showed significant contrast in T1, T2 and T2* weighted images immediately after injection. As expected, T2 and T2* images showed a much more drastic effect than T1, but the T1 effect is nevertheless significant. The contrast was quite drastic as seen in the chosen T2 scan, where negative contrast can be observed throughout. Massive uptake by the reticuloendothelial system (RES) is immediately evident, especially in the liver and spleen where dark areas are seen after injection. Signal Intensities in the liver, kidneys and spleen were also measure before, 1 h and 2 h after injection. The results again show signal intensities loss of 70% to 90%, which show rapid uptake by liver and spleen while signal loss in the kidneys reflects its presence in circulation. Presence in circulation was determined by signal intensities in the kidneys, size restriction prevents renal clearance and absence of negative contrast in urether support this claim. Presence in circulation using this method seem to infer that MCL are still in circulation after 24 h and even after 48 h, but more accurate methods such as angiography at such time points are needed.

This initial study demonstrated that the concentration of magnetite (20 mg/kg-25 mg/kg) is sufficient to show exceptional negative contrast on T1, T2, and T2* images. Although this concentration is high as compared to concentrations used for assessing lymph nodes, the efficiency of contrast enhancement cannot be compared directly to such functionalities. In this application contrast is needed to track drug carriers and monitor treatment rather than detecting lymph node metastases and other diagnostic capacities. Even though the concentration of magnetite was high, no adverse effects were observed in the animals.

Tumor-Bearing Mice

Following analyses of healthy animal study, T2 and T1 scans were chosen as best scans in terms of contrast and scan time. In addition a T2* sequence of scans (12.6/2763, 5-acquisitions, 90° flip angle) was chosen to obtain T2* maps. The next study involved 16 10-week-old SCID mice with a carcinoma tumor grown on the posterior right flank for 10 days. The 16 mice were divided into 4 subgroups: 8 injected intravenously, 4 with magnet applied for 1 h and 4 without, and 8 injected intratumorally, 4 with magnet applied for 1 h and 4 without. All mice were imaged before and 24 h after injections using previously chosen scans and T2* values determined on 2 of each subgroup.

The group of intravenous injections without magnet again showed massive uptake by RES with some accumulation in the tumor. This accumulation varied according to tumor size and homogeneity; tumors that are in smaller pieces with space in between are more susceptible to accumulating MCL in this space with some diffusion through the tumor bulk. Such accumulation is only evident in tumors that are highly inhomogeneous and in multiple pieces. All the tumors from this group were not very solid, but comparing the two animals shown one can infer the effects of such inhomogeneity.

The group of intravenous injection with magnet applied for one hour consistently showed remarkable signal decrease in tumor, indicating great MCL accumulation. The magnet was applied during the first hour post-injection while imaging was done 24 h post-injection, demonstrating the ability to not only target MCL towards the tumor, but to retain such accumulation. One animal was chosen to be imaged after 48 h to further investigate MCL retention. The results clearly show the remarkable accumulation and retention even after 48 h.

Quantitative analysis of tumors was also done by choosing tumor areas in the bulk and calculating the average signal intensities. The average signal intensity of adjacent muscle areas were also chosen as a reference and the tumor/muscle ratio was calculated for all animals. This ratio was used to obtain a percent signal decrease before and after injection. In the case for magnetically targeted tumors, the area directly adjacent to where the magnet was placed was treated separately since inhomogeneous magnetic force targets mostly this area. This area was referred to as the maximum target area and quantified separately from the rest of the tumors. The analysis results in an average decrease in tumor/muscle ratio of −19.72%±5.18% for the non-magnet group; this compares to −56.87%±12.66% in maximum target area of magnet group. The area not included in the maximum target area of the tumor showed an average −10.14%±8.57% in the tumor mice.

In addition to the tumor/muscle ratio calculated, two animals from each group were used to calculate T2* values before and after injection. These values were obtained choosing small square regions of interest (<3 mm²) in desired area of the T2* maps obtained. The results show the average decrease of T2* values in the tumor, liver and kidneys. For the intravenous group there is a two-fold decrease in T2* percent loss which demonstrates greater accumulation with magnet than without. This supports results obtained comparing signal intensities. In addition the T2* values in the liver and kidney show decreased presence of MCL in both organs with magnet than without. This could reflect the increased MCL accumulation in the tumor leads to less uptake by the liver.

### 14. Summary of Experimental Results

MAG-C cationic liposomes can incorporate MAG-C, maintain a cationic charge potential in presence of magnetic material, are taken up by cancer and endothelial cells, and under certain conditions can respond to an external magnet in vitro and in vivo. These characteristics are helpful to overcome heterogeneous vascular targeting with cationic liposomes when used to induce tumor vascular injury with vascular disrupting agents. These experimental findings support the use of MAG-C cationic liposomes as a carrier for the delivery and prolonged tumor accumulation of chemotherapeutic agents in the presence of an external magnetic field. These liposomes are highly efficacious for use in MRI for monitoring therapeutic benefit. They can also be used to deliver drugs through sustained release and on-demand or triggered release.

### Example 2

### 1. Liposome Size

Changes in liposome size and dispersity were important indicators of change in bilayer properties of liposomes. Cholesterol significantly reduced liposome diameter of DMPC/DMTAP liposomes from 369±144.38 run to 105±26.64 nm. When SPION (SPION) was added at 0.5 mg/ml there was no significant change in mean diameter of DMPC/DMTAP/cholesterol liposomes, but higher SPION content (2.5 mg/ml) resulted in a significant increase in mean diameter from 105±26.64 nm to 267±27.43 nm.

### 2. Zeta Potential

Quantitative changes in surface charge characteristics of liposomes due to the inclusion of different liposome components can be used to predict the relative affinity of tumor vessels for liposomes on the basis of charge. Zeta potential values were determined immediately following size measurements, and were therefore the identical preparations used for the determination of size. Zeta potential for electroneutral liposomes consisting of DMPC alone was −0,26±3.99 my. This value increased significantly in the presence of 50 mol % DMTAP (84.96±15.57 my; P<0.05). The inclusion of 10 mol % cholesterol in DMPC/DMTAP preparations reduced zeta potential from 84.96±15.57 to 64.55±16.68 (P>0.05). The inclusion of 0.5 mg/ml of SPION into cationic liposomes significantly reduced the zeta potential of MCL (43.79±10.93 mv) compared to preparations without SPION (64.55±16.68 mv). An increase in SPION (2.5 mg/ml) content resulted in no significant changes in zeta potential compared to preparations containing 0.5 mg/ml of SPION.

### 3. Iron Content

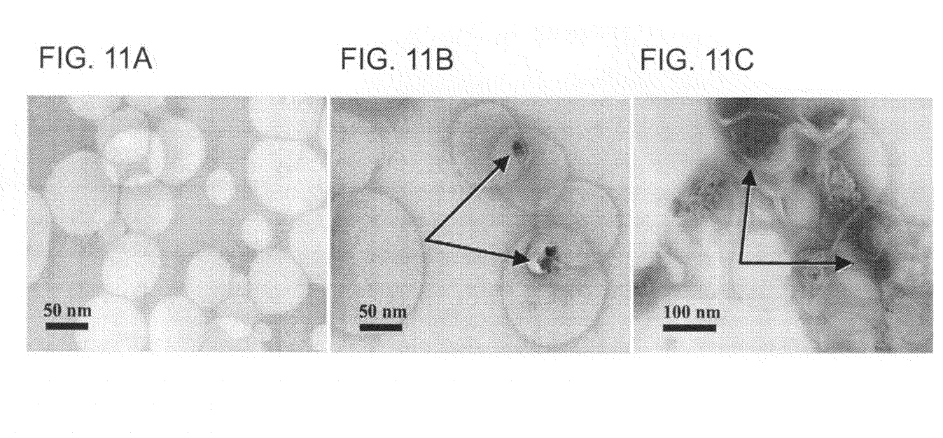

The fraction of SPION loaded must be sufficient to respond to an external magnetic field and to provide strong MRI contrast enhancement (Alexiou 2000; Babincova 2000). Therefore, the amount of iron incorporated in each liposome variety was determined spectrophotometrically. Starting with 1.8 mg/ml of iron content (from the 2.5 mg/ml of SPION liposome preparations), the amount of iron incorporated in electroneutral liposomes (EL), cationic liposomes (CL), and Pegylated-CL was 0.4170.12, 1.1770.17, and 1.5370.2 mg/ml, respectively. TEM images confirmed association of SPION with cationic liposomes (FIGS. 11A-C). Moreover, TEM showed relatively high association of SPION with cationic liposomes containing 2.5 mg/ml compared to 0.5 mg/ml.

### 4. Cell Viability Assay

No toxic effects on cell growth were observed against murine melanoma (B16-F10), human melanoma (HTB-72), and human dermal microvascular endothelial (HMVEC-d) cells at <1000 nmol (Dandamudi 2007a). The percent of viable B16-F10, HTB-72 and HMVEC-d cells after 24 h exposure to 500 nmol of liposomes was 92, 85, and 78% respectively. Although the values reported here for HMVEC-d tended toward relatively lower cell viabilities, no statistically significant effects were observed. The percent of viable cells remaining in culture after exposure to SPION cationic liposomes reduced significantly at ≧500 nmol compared to 10 nmol with all three cell lines. Moreover, we observed no significant difference in cell viability among cell lines at any given concentration, suggesting that the liposomes were equally toxic to both cancer and microvascular endothelial cells.

### 5. Cell Association Studies

The tendency of SPION cationic liposomes to associate with cells increased with liposome amount, and association values were similar for the three cell lines. No significant increase was observed in cell association >500 nmol of liposomes (Dandamudi 2007a). DIC and fluorescence microscopic imaging showed avid accumulation of SPION cationic liposomes evident in all cell lines with the most significant uptake observed in B16-F10 and HTB-72. In addition, perinuclear uptake was noted in HMVEC-d cells.

### 6. Role of PEG in MCL

The inclusion of DMPE-PEG to the cationic liposome preparation containing relatively high SPION (2.5 mg/ml) content resulted in a significant decrease in liposome size from 262.67±27.43 to 156±16.25 nm. The interaction of PCLs with cancer (B16-F10 and HTB-72) and endothelial (HUVEC) cell lines was compared. The inclusion of PEG significantly reduced cellular interactions regardless of cell type. These data suggest that the inclusion of 5 mol % PEG altered bilayer physical properties such as liposome cationic surface charge potential, but did not affect liposome size. In vivo MRI studies clearly established the long circulation properties of pegylated MCL. Significant contrast enhancement was observed in the blood pool 48 hours after intravenous administration, indicating that MCL are present in the blood pool for a few days (Gultepe 2009b).

### 7. Magnetic Susceptibility

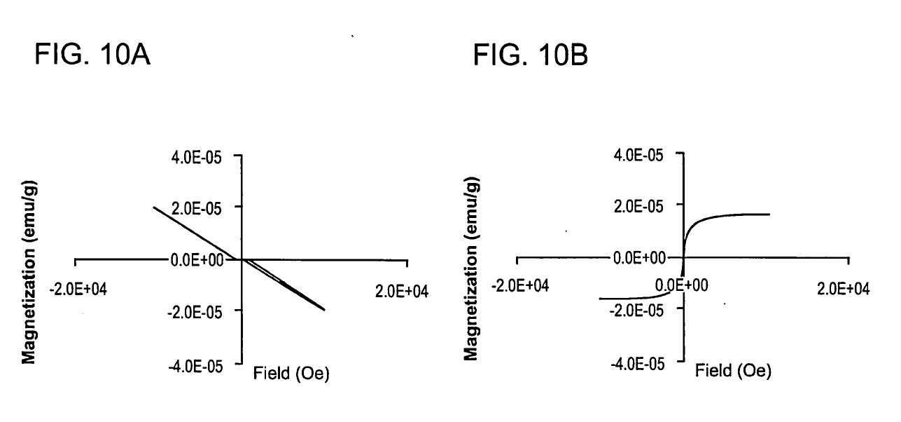

FIG. 10 displays the magnetization curve as a function of the magnetic field for each preparation type. Cationic liposomes (without SPION) showed the typical curve for diamagnetic materials confirming a lack of magnetic susceptibility (FIG. 10A). The magnetization curves of MCL (FIG. 10B) did not show a hysteresis loop confirming the superparamagnetic behavior of SPION. The saturation magnetization value of magnetic cationic liposomes containing 2.5 mg/ml of SPION (4.8*10-3 emu/g of lyophilized liposome) was significantly higher than 0.5 mg/ml (1.6*10-3 emu/g of lyophilized liposome). Our values for magnetic susceptibility correlated with the quantity of iron measured in each preparation (Dandamudi 2007b).

8. In vivo Tissue Distribution Profile of ¹¹¹In Labeled SPION Cationic and PEGylated MCL

The effect of PEG on the tissue distribution profile of MCL in melanoma bearing SCID mice was determined. When MCL were injected intravenously into melanoma bearing mice, the percent of injected dose per gram of the tissue in lung, liver and spleen was 35.27, 56.61 and 75.92% respectively. The inclusion of 5 mol % DMPE-PEG resulted in a significant reduction in the uptake by the major organs of RES. The significant reduction was due to the electrostatic repulsive effect of PEG (Torchilin 1995; Ishida 2002). Moreover, tumor/blood ratio for PEGylated MCL (4.36±1.14) was significantly higher than that for MCL (1.56±0.43). The data suggest that PEG minimizes uptake of MCL by healthy tissues while improving tumor uptake.

Tumor accumulation of MCL in presence of external magnet enhanced tumor vascular association is the preferred mechanism of uptake given the natural affinity of cationic liposomes for tumor vessels (McLean 1997; Thurston 1998; Campbell 2002). Vascular accumulation can be further enhanced by application of magnetic targeting. We have studied tumor accumulation in two ways—using fluorescently labeled MCL in a dorsal skin-fold animal model, and with radio-labeled MCL (FIG. 6). Both methods show substantial enhancement of accumulation due to magnetic targeting.

9. Drug Loading into MCL

The influence of SPION on the efficiency of loading chemotherapeutic agents (hydrophilic and lipophilic) in cationic liposomes was evaluated by HPLC. We evaluated the effect of SPION on the incorporation efficiency of the hydrophilic drug, dacarbazine at 3mol %. The addition of SPION at lower concentrations (0.5 mg/ml) did not influence the incorporation efficiency of dacarbazine (28±3.02%), efficiency of loading however decreased from 28±3.02% to 19±3.2% with 2.5 mg/ml SPION. As observed with etoposide and vinblastine sulfate, PEG significantly improved the incorporation of dacarbazine in cationic liposomes from 19±3.2% to 30±1.58%.

Gamma imaging We evaluated the effect of the external magnet (1.2 T, I h) in vivo on tumor uptake of MCLs in melanoma-bearing SCID mice. Nobuto et al. (Nobuto 2004) have studied the application of magnetic fields on the accumulation of doxorubicin in tumors. The authors showed that an increase in the application time of the electromagnetic field (up to 60 min) increased the concentration of doxorubicin within the tumor. However, no additional increase in tumor doxorubicin concentration was observed with 100-min application of magnetic field. In the present study, the external magnet was placed for 1 h. We observed a greater signal in tumors previously exposed to the external magnet (exposure time, 1 h) compared to the no-magnet group (FIG. 7). The use of an external magnet thus improved the accumulation and retention of MCLs in the tumors.

### 10. Efficacy Evaluation of Vinblastine-Loaded MCL

In mice treated with free vinblastine sulfate (1.35 mg/kg), no significant difference in the tumor volumes when compared to the untreated control group was observed. The magnetic field enhanced the tumor response to the formulation when compared to free vinblastine and untreated control group. When the external magnet was applied, the tumor volumes remained relatively constant (no increase in overall volume when compared to day 1 of treatment). On day 16 there was a significant difference in the antitumor effects of vinblastine-loaded MCLs in the presence of an external magnet compared to no magnet (a). The external magnet reduced the tumor volume (131 □37 mm3) to a significantly greater extent compared to no magnet (227 □22 mm3). At the end of the experiment, the percent of change in tumor volume was significantly lower for the formulation (24 □32%) in the presence of a magnet when compared to the no-magnet (94 21%), free vinblastine (270 177%), and untreated (378 185%) groups (FIG. 8).

Tumor metastases Melanoma tumors originate from melanocytes and produce excessive melanin pigmentation (Das 1975). The presence of melanin pigmentation in the different organs was used to track tumor metastasis, since under normal conditions melanin is not present in healthy SCID mice. The primary sites of melanoma metastases are lung, liver, lymph nodes, spleen, brain, and intestines (Murakami 2004). The H&E images of liver and spleen showed no significant differences in nuclei staining between the different treatment groups. We found relatively high melanin pigmentation in the liver and spleen of the saline control group (FIG. 9a). This would suggest that the primary tumor metastasized to these organs. The number of tumor nodules was less in the livers of mice treated with free vinblastine sulfate compared to the saline control. We also found melanin pigmentation in tissue sections of mice treated with vinblastine-loaded MCLs in the absence and presence of an external magnet, but relatively less compared to the other treatment groups. All mice (5/5) in the saline- and free-vinblastine-treated groups showed evidence of metastasis within the pleural cavity (FIG. 9b[i, ii]). In the group treated with vinblastine-loaded MCLs having no exposure to the magnet, two out of five (40%) mice showed no signs of metastasis, and the remaining three mice in group showed relatively less in comparison to the saline control and free vinblastine groups (FIG. 9b[iii]). On the other hand, we did not observe any signs of metastasis in the pleural cavity of mice in the magnet group (FIG. 9b[iv]). The external magnet probably retained a significantly higher fraction of the drug within the tumor, severely compromising the tumor's ability to metastasize to the pleural cavity. The s.c. injection of B16-F10 cells did result in the formation of detectable lung metastases within the first 16 days; however, regardless of the treatment group, no melanin pigmentation was observed in the lung parenchyma of any mouse in study. This is unlike studies involving the intravenous injection route, here the aggressive cellular phenotype rapidly forms tumor nodules within the lung parenchyma.

11. MRI Imaging with MCL

MR imaging was performed on a 7 T preclinical MRI system (BioSpec 70/20USR, Bruker BioSpin Corp., Billerica, Mass.) at the Center for Translational Neuro-Imaging at Northeastern University. A whole body quadrature coil was used for reception and emission.

Healthy mice A study of 10-week-old healthy SCID mice was carried out to determine the best imaging sequence and parameters to obtain optimal contrast from SPION. The study on healthy animals showed significant contrast in T1, T2 and T2* weighted images immediately after injection. As expected, T2 and T2* images showed a much more drastic effect than T1, but the T1 effect is nevertheless significant. The contrast was quite drastic as seen in the chosen T2 scan shown in FIG. 10, where negative contrast can be observed throughout. Massive uptake by the reticulo endothelial system (RES) is immediately evident, especially in the liver and spleen where hypointense areas are seen after injection. Signal Intensities in the liver, kidneys and spleen were also measured before, 1 hour and 2 hours after injection. The results in FIG. 10 again show signal intensities loss of 70% to 90%, which show rapid uptake by liver and spleen. Presence in circulation was determined by signal intensities in the kidneys, size restriction prevents renal clearance and absence of negative contrast in the urether support this claim. Presence in circulation using this method seems to infer that MCL are still in circulation after 24 hours and even after 48 hours. This initial study demonstrated that the concentration of SPION (20-25 mg/kg) is sufficient to show exceptional negative contrast on T1, T2, and T2* images. One must note that even though the concentration of SPION was quite high, no adverse effects were observed in the animals.

### 12. Tumor-Bearing Mice

The next study involved 16 10-week-old SCID mice with a carcinoma tumor of metastatic melanoma cancer cells (B16-F10) grown on the posterior right flank for 10 days. The 16 mice were divided into four subgroups: 8 injected intravenously, 4 with magnet applied for 1 hour and 4 without and 8 injected intratumorally, 4 with magnet applied for 1 hour and 4 without. All mice were imaged before and 24 hours after injections using previously chosen scans and T2* values determined on 2 animals of each subgroup. Pre-injection and post-injection MR images were used to assess response to magnetic targeting effects (Gultepe 2009b).

Hypointense areas in MR images, decreased signal intensity and lower T2* relaxation times all directly correlate to MCL accumulation. We have shown that tumor signal intensities in T2 weighted images decreased an average of 20±5% and T2* values decreased and average of 14±7 ms in the absence of MT. This compares to an average signal decrease of 57±12% and a decrease in T2* relaxation times of 27±8 ms with the aide of MT, demonstrating excellent MRI contrast capabilities, a higher accumulation and retention of MCL in magnetically targeted tumors.

For the IV injection groups, the difference in tumor accumulation of MCL between the groups with or without applied field is quite clear in the MR images (FIG. 11). With magnetic guidance, there is a remarkable signal decrease in the tumor, indicating greater MCL accumulation with magnet than the no magnet group. Diffusion through tumor bulk with magnetic guidance was more efficient than the group without. The uniform darkening of the target area illustrates the effect of the external magnet where a more homogeneous accumulation is achieved as compared to non magnet group.

The magnet was applied during the first hour of injection while imaging was done 24 hours after the injection. Hence, the images demonstrate the ability to not only target MCL towards the tumor, but to retain such accumulation.

Signal Intensity analysis for IV injection group showed almost 3-fold enhancement in the contrast for the magnetic guidance group comparing non-magnet group as seen in FIG. 11. T2* values show 2-fold decrease in the values for tumor comparing magnet and non magnet group. This decrease in T2* values corresponds to a 2-fold increase in the accumulation of MCL inside the tumor with the presence of the applied field, FIG. 11. T2* maps shown in FIG. 11 reflect a spatial distribution of T2* values before injection and after injection. In animal shown from group IV-M significant decrease in T2* values in the bulk of the tumor is evident where T2* values went from approximately 60 ms before injection to as far down as 30ms after injection. In the animal shown from group IV-NM heterogeneous accumulation around tumor bulk is observed throughout.

### 13. Tumor Distribution of Magnetic Cationic Liposomes

Intratumoral accumulation in group IV-NM varied according to tumor homogeneity and its size as shown in FIG. 11. Tumors that are highly heterogeneous were more susceptible to accumulating MCL in surrounding space with some diffusion through the tumor bulk. Such effect can be observed on all three animals from group IV-NM. It should also be noted that MCL accumulation in group IV-NM is heterogeneous as we have shown it's the case for cationic liposomes. In group IV-M penetration through tumor bulk was far more drastic than IV-NM. Diffusion through tumor bulk with magnetic guidance was more efficient than without, uniform darkening of maximum target area illustrate the effect of the external magnet. This demonstrates that magnetic targeting can be effectively used to obtain a homogenous accumulation of MCL in tumor.

### Example 3

Following IV administration of between 0.1 mg/kg to 10 mg/kg magnetic nanoparticles (“MNL”) comprising SPION, the MNL accumulate in a tumor mass. MNL formulations are administered in concentrations from 2-25 mg/mL. The tumor mass is exposed to external oscillating magnetic fields and temperatures are maintained at 45° C. for fixed time intervals. Subsequently, the tumor is irradiated with 200 keV Xrays in a Small Animal Radiation Research Platform. Any change in tumor mass and survival rates of tumor-bearing animals after treatment as a function of applied magnetic field strength and exposure time is compared with results for controls. Histologic studies of tumor necrosis after animal sacrifice is carried out.

The results show that the mice receiving the MNL and hyperthcmal therapy have smaller tumor mass and survival rates compared to controls.

### Example 4

The focus of this example is to study loco regional hyperthermia followed by radiation therapy in vivo in PC-3 prostate cancer model in nude mice.

### 1. Production of MNL

For all the studies in this project, MNL are made with DPPC:DOTAP:CHOL and DOPE-PEG5000, with lipid ratios (60:20:15:5), containing 15 mg/mL of SPION, and labeled with the fluorescent dye rhodamine.

2. In vitro Studies of Targeting Efficiency of MNL

The targeting efficiency of our fluorescent MNL in vitro using the PC3, human prostate cancer cell line. The in vitro studies dealing with the theranostic effects of these fluorescent MNL on prostate (PC-3s) cancer cell lines have two components, (a) imaging, and (b) therapy. For the imaging studies, the cells treated with MNL are analyzed for up to 24 hours post-treatment. The imaging studies utilize confocal fluorescent microscopy to track the fluorescent dye rhodamine and are used to optimize liposomal formulation for maximum uptake in target cells. Toxicity studies follow, probing the cytotoxic activity of the magnetic heating, using the MTT assay. For these studies, the cells are subjected to heating at hyperthermic temperatures of 45° C., and ablative temperatures of >60° C., for 30 minutes each, and incubated for 24 hours. Cytotoxicity of fluorescent MNL are assessed as a function of concentration using the 3-(4, 5-dimethylthiazol-2-yl)-2,5-diphenyltetrazolium bromide dye reduction (MTT) assay on the PC3 cell lines. The assay is carried out using standard 96-well microplates according to manufacturer's instructions (Biotium, California, USA). Cell proliferation is assessed after incubation (24 hours) with media containing varying concentrations of liposomes. The uptake and distribution of fluorescent MNL in cultured cells are assessed using NIR optical imaging. Following exposure to varying concentrations of MNL, tissue culture plates are imaged using a Keck 3D Fusion Microscope. Experiments are performed in triplicate and 1050 values calculated.

3. Study in vitro Effect of Xray Irradiation Following Thermal Therapy of PC-3 Cell Lines.

The effect of x-ray irradiation are studied following thermal therapy PC-3 cell lines. These experiments test the hypothesis that thermal therapy enhance the radiation response of this PCa cell line. Cell lines are incubated with MNL and are subjected to ac magnetic fields to raise temperatures up to 45° C. for 30 mins. Subsequently the cells are irradiated with graded doses (1-10 Gy) 200 keV X-rays using a small animal radiation research platform. Cell proliferation are assessed for the following groups: (i) no treatment, (ii) TT treatment for 30 mins, (iii) TT treatment for 60 mins, TT treatment for (iii) 30 min, and (iv) 60 mins, then followed by RT each. Experiments are performed in triplicate and IC50 values calculated.

### 4. Tumor Model Development

PC3 are established in female athymic nude mice. PC3 cells (1X105) are injected in the hind flank region of the mice under anesthesia. Periodically, the animals are observed for tumor growth by palpation. The PIs lab has the expertise to utilize PC3 model for evaluation of various anticancer drug delivery systems.

5. In vivo Biodistribution Studies

Studies are carried out to quantify MNL plasma half-life, organ biodistribution, and tumor uptake (i) without and (ii) with magnetic targeting, in healthy and tumor-bearing mice.

BioSpec 70/20USR (Bruker BioSpin Corp., Billerica, Mass.) MRI scanner at the Center for Translational Neuro-Imaging at Northeastern University are used for the MRI studies. Quantitative analysis of MR images are performed by using either ParaVision or additional software like MATLAB and MIVA (Medical Image Visualization and Analysis Software). For a successful MR analysis, each animal is imaged prior to administration of any agent. Pre-imaging provides a baseline for SI values or parameter maps. The studies are performed both in the absence and presence of the permanent magnet, prior to MRI evaluation. Approximately 8 mice per/group are used to determine contrast enhancement in the tumor groups, and signal intensity are plotted as a function of SPION content to determine optimal concentration (See Table 2).

In vivo biodistribution and pharmacokinetics of fluorescent MNL encapsulating SPION and rhodamine are evaluated. Dose escalation studies are performed to assess tolerance (toxicity) and biodistribution using NIR optical imaging and MRI. To determine the kinetics of elimination of the contrast agent, serial MRI/OI are carried out at different times post contrast agent administration in naïve and tumor-bearing mice (n-5 per group). For tumor targeting an external magnet are used. Concentration of the nanoparticles are obtained in tumor, normal tissues and in plasma at different times following administration. Pharmacokinetic parameters including distribution half life, plasma clearance, elimination rate constant, plasma to tissue rate constant are assessed. After systemic injection of the fluorescent MNL, the in vivo optical as well as MR imaging are used for visualizing the localization of the liposomes in the mice. The fluorescence of the rhodamine is monitored optically whereas the SPIONs incorporated in liposomal matrix are tracked by MRI. Imaging parameters (T2-enhancement) are used as an indirect ‘read-out’ of localization of liposomes and correlated with intratumoral concentrations of rhodamine using fluorescence and HPLC analysis.

In addition to standard measures of toxicity (change in body weight, signs of morbidity), histopathological assessment of normal tissues (heart, lungs, liver and brain) are performed (n=4/group). The toxicity profiles of MNLs are evaluated using histologic analysis of normal and tumor tissues.

### 6. In Vivo Evaluation of Combined Hyperthermic and Radiation Therapy:

The therapeutic efficacy of the MNLs is determined, with applied alternating magnetic field, followed by radiation therapy. In a typical experiment, mice bearing subcutaneous prostate tumor xenografts are injected systemically by intravenous route with different MNL formulations (as described in table 1) at an optimized dose. In addition, one ‘arm’ of control mice each receives injection of PBS only, at the same time points post-implantation. Therapeutic studies are carried out following establishment of the optimal dose/schedule of MNLs administration. For assessing the therapeutic response, the best optimized MNL formulation that has shown promise in tumor targeting in the previous studies is selected. Once the tumor develops to a size of 10 mm in diameter, a total of 96 animals are divided into groups of 8 each for the following control and test experiments:

The MNL, suspended in saline, are injected intravenously through the tail vein. At various time points, the animals are treated with alternating magnetic field in groups for 30 mins and I hour. Temperature changes in the tumor are monitored with an accurately-calibrated infrared thermocouple device. The temperature is manually controlled to be maintained at 45 OC (for hyperthermia). The hyperthermia therapy is followed by radiation therapy using the SARRP. A single-fraction radiation The radiation dose of 5 Gy or, alternatively, 15 Gy are administered to the mice following hyperthermia. selected based on the results of the in vitro studies with PC3 cells.

The tumor volume measurements are performed daily by measuring the three orthogonal diameters of the tumor and calculating the volume using the equation: Tumor Volume (in mm3)=(□/6)(D1D2D3), where D1, D2, D3 are the three orthogonal diameters (in mm). Also, Changes in primary tumor and lymph node volume are assessed using MRI/OI.

In addition, the weight of the tumor mass at the time of sacrifice (typically 8-10 days post-administration) is measured. Tumor samples are sent to Charles River Laboratories (Wilimington, Mass.) for histological analysis of apoptosis or necrosis following hyperthermia, radiation and thermal ablation treatments. Lastly, the percent of tumor bearing animals that survived the control and test treatments until the time of sacrifice are determined as well. If the tumor burden has exceeded 15% of body weight during the study and at the end, the animals are euthanized by CO₂ inhalation. Animals are monitored for two months for tumor growth using whole body MRI and OI, followed by their sacrifice and post-mortem analysis.

Table 3 summarizes the groups in in vivo studies.

TABLE 3

| | # | | Radiation | HT + |
|---|---|---|---|---|
| Batch of animals | animals | Hyperthermia | therapy | RT |
| PBS saline | 8 | No treatment (control) | | |
| | 8 | Hyperthermia | | |
| | 8 | | Radiation therapy | |
| | 8 | | | Both |
| MNL Non Targeted | 8 | Hyperthermia | | |
| | 8 | | Radiation therapy | |
| | 8 | | | Both |
| MNL Targeted | 8 | Hyperthermia | | |
| | 8 | | Radiation therapy | |
| | 8 | | | Both |

### EQUIVALENTS

Those skilled in the art will recognize, or be able to ascertain, using no more than routine experimentation, numerous equivalents to the specific embodiments described specifically herein. Such equivalents are intended to be encompassed in the scope of the following claims.

1-14. (canceled)

15. A method of multi-modal diagnostic imaging, the method comprising:
(a) administering a nanoparticle composition to a subject, the nanoparticle composition comprising three or more members selected from the group consisting of paramagnetic particles, radiolabels, fluorophores, and positron emission tomography agents encapsulated within a biocompatible vehicle;
(b) allowing the nanoparticle composition to bind to a tissue or to circulate in the vasculature in the subject;
(c) detecting the nanoparticle composition by one or more imaging techniques selected from the group consisting of positron emission tomography (PET), magnetic resonance imaging (MRI), single photon emission computed tomography (SPECT/CT), and optical imaging;
(d) generating one or more images of the tissue bound by the nanoparticle composition or of the circulation system, and
(e) registering the images from different modalities to obtain accurate location of the tissue and or the organs being imaged.

16. The method of claim 15, wherein the nanoparticle composition is about 30 nm to about 250 nm.

17. The method of claim 15, wherein the biocompatible vehicle is a micelle.

18. The method of claim 15, wherein the biocompatible vehicle is a liposome.

19. (canceled)

20. The method of claim 15, wherein the one or more paramagnetic particles are superparamagnetic iron oxide nanoparticles.

21. The method of claim 15, wherein the biocompatible vehicle further comprises polyethylene glycol.

22. (canceled)

23. The method of claim 15, wherein the biocompatible vehicle further comprises one or more targeting agents.

24. The method of claim 23, wherein the one or more targeting agents are antibodies or binding fragments thereof

25-40. (canceled)

41. The method of claim 15, wherein the biocompatible vehicle further comprises siRNA molecules.

42. The method of claim 41, wherein the siRNA is SEQ ID NO: 1.

43. A method of treating diseased tissue by magnetic heating, the method comprising:
(a) administering a magnetic nanoparticle composition comprising paramagnetic particles and one or more members selected from the group consisting of radiolabels, fluorophores, and positron emission tomography agents, the paramagnetic particles and one or more members being encapsulated within a biocompatible vehicle, the nanoparticle composition being about 30 nm to about 250 nm;
(b) allowing the nanoparticle composition to localize to a diseased tissue in the subject, and
(c) subjecting the nanoparticle composition to an alternating (ac) magnetic field such that the temperature of the nanoparticle composition and attached tissue increases to 42-45° C. for hyperthermic treatment, and above 45° C. for thermal ablation; thereby killing the diseased cells in the tissue.

44-66. (canceled)

67. A method of treating diseased tissue, the method comprising:
(a) administering a nanoparticle composition comprising paramagnetic particles and siRNA molecules encapsulated within a biocompatible vehicle;
(b) allowing the nanoparticle composition to localize to a diseased tissue in the subject, the siRNA molecules being released into the cells of the diseased tissue so that the diseased tissue is treated with the siRNA.

68. The method of claim 67, wherein the biocompatible vehicle encapsulates paramagnetic particles, siRNA molecules and two or more of the group consisting of radiolabels, fluorophores, and positron emission tomography agents

69. The method of claim 68 further comprising detecting the nanoparticle composition by one or more imaging techniques selected from the group consisting of positron emission tomography (PET), magnetic resonance imaging (MRI), single photon emission computed tomography (SPECT/CT), and optical imaging.

70. The method of claim 68, wherein the siRNA is SEQ ID NO: 1.

71. The method of claim 67, wherein the nanoparticle composition is about 30 nm to about 250 nm.

72. The method of claim 67, wherein the biocompatible vehicle is a micelle.

73. The method of claim 67, wherein the biocompatible vehicle is a liposome.

74. (canceled)

75. The method of claim 67, wherein the one or more paramagnetic particles are superparamagnetic iron oxide nanoparticles.

76. The method of claim 67, wherein the biocompatible vehicle further comprises polyethylene glycol.

77-94. (canceled)
